(19) United States
(12) Patent Application Publication
(10) Pub. No.: US 2014/0255341 A1
(43) Pub. Date: Sep. 11, 2014

(54) Tumor Selective Chemokine Modulation

(71) Applicants: Pawel Kalinski, Wexford, PA (US); Ravikumar Muthuswamy, Monroeville, PA (US)

(72) Inventors: Pawel Kalinski, Wexford, PA (US); Ravikumar Muthuswamy, Monroeville, PA (US)

(21) Appl. No.: 14/234,026

(22) PCT Filed: Jul. 23, 2012
(86) PCT No.: PCT/US12/47887

§ 371 Date: Jan. 21, 2014

Related U.S. Application Data
(60) Provisional application No. 61/510,855, filed on Jul. 22, 2011.

(51) Int. Cl. A61K 38/21; A61K 31/713; A61K 45/06; A61K 31/415; A61K 31/197; A61K 31/405; A61K 31/517; A61K 35/26

(52) U.S. Cl. CPC A61K 38/212; A61K 31/517; A61K 31/713; A61K 35/26; A61K 31/415; A61K 31/197; A61K 31/405; A61K 45/06
USPC 424/85.2; 424/85.7; 514/266.4; 514/44 R; 424/184.1; 424/172.1; 424/85.1; 424/85.6; 424/85.5

(57) ABSTRACT

Therapies effective for the treatment and prevention of cancer and other diseases are disclosed. These methods include the administration of therapeutically effective amounts of agents that increase the local production of effector cell-attracting chemokines within tumor lesions, with concomitant suppression of local production of undesirable chemokines that attract regulatory T(reg) cells. These methods include administering to the subject therapeutically effective amounts of a Toll-like receptor (TLR) agonist or other activator of NF-KB pathway in combination with a blocker of prostaglandin synthesis or a blocker of prostaglandin signaling, in combination with a type-1 interferon, or in combination with both a blocker of prostaglandin synthesis or signaling and with a type-1 interferon. Alternatively, the methods derived from the same paradigms, but aimed to treat or prevent autoimmune disease, chronic inflammatory disease, transplant rejection or GvR, include the combination of a Toll-like receptor (TLR) agonist in combination with a prostaglandin or other cAMP-activator.

### STATEMENT OF PRIORITY

This claims the benefit of U.S. Provisional Application No. 61/510,855, filed Jul. 22, 2011, which is incorporated by reference herein.

### STATEMENT OF GOVERNMENT SUPPORT

This invention was made with United States government support pursuant to grant 1PO1 CA132714 from the National Institutes of Health; the United States government has certain rights in the invention.

### FIELD

This is related to the field of tumor therapy including the methods for the treatment of cancer, prevention of cancer occurrence or prevention of cancer recurrence. In addition, the described methods may be used in the treatment of infectious diseases, autoimmune diseases, allergies and inflammation, as well as to treat or prevent transplant rejection and transplantation-associated disorders.

### BACKGROUND

Each year in the United States alone, untold numbers of people develop precancerous lesions, a form of neoplasia, as discussed below. Such lesions exhibit a strong tendency to develop into malignant tumors, or cancer. Such lesions include lesions of the breast (that can develop into breast cancer), lesions of the skin (that can develop into malignant melanoma or basal cell carcinoma), colonic adenomatous polyps (that can develop into colon cancer), premalignant lesions of the cervical epithelium (that can develop into cervical cancer) and other such neoplasms. Compounds that prevent or induce the remission of existing precancerous or cancerous lesions or carcinomas, or to prevent or delay their recurrence following treatment by surgery, chemo-, radio-, and biologic therapies, would greatly reduce illness and death from cancer.

For example, approximately 60,000 people die from colon cancer, and over 150,000 new cases of colon cancer are diagnosed each year. For the American population as a whole, individuals have a six percent lifetime risk of developing colon cancer, making it the second most prevalent form of cancer in the country. Colon cancer is also prevalent in Western Europe. It is believed that increased dietary fat consumption is increasing the risk of colon cancer in Japan.

To date, little progress has been made in the prevention and treatment of advanced cancer, including colorectal cancer, ovarian cancer, prostate cancer, breast cancer, lung cancer, head and neck cancer, cervical cancer, brain cancer, pancreatic cancer, kidney cancer, hematologic malignancies and melanoma, as reflected by the lack of change or only moderate change in the five-year survival rate over the last few decades. The only cure for this cancer is surgery at an extremely early stage. Unfortunately, most of these cancers are discovered too late for surgical cure. In many cases, the patient does not experience symptoms until the cancer has progressed to a malignant stage.

The ability of CD8⁺ T cells to infiltrate cancer lesions is essential for anti-tumor immunity, as evidenced by studies highlighting the prognostic value of effector T (T_{eff}) cells in multiple cancer types, including colorectal cancer (CRC), ovarian cancer and melanoma. In contrast, infiltration of different types of cancers with regulatory T cells (T_{regs}) predicts poor outcomes. Chemokines and their respective receptors are critical for T cell migration and homing.

The data from several groups have demonstrated that different groups of chemokines (CKs) preferentially attract either pro-inflammatory immune cells: effector T(eff) cells (CTLs, Th1 cells) and NK cells, desirable in cancer and chronic infections, or regulatory T(reg) cells, believed to be detrimental in cancer and chronic infections. Pro-inflammatory immune cells express high levels of CXCR3 and CCR5 (chemokine receptors for, respectively, CXCR9, CXCR10, CXCR11, and for CCL3, CCL4 and CCL5 and CCL5), while Tregs express high levels of CCR4 and CXCR4. Tregs preferentially express CCR4 (receptor for CCL17 and CCL22), CXCR4 (receptor for CXCL12), and CCR6 (receptor for CCL20).

High levels of CCL5/RANTES (CCR5 ligand) and CXCL9/MIG and CXCL10/IP10 (ligands for CXCR3) in tumor tissues are associated with enhanced infiltration of CD8⁺ T cells in CRC, melanoma and gastric cancer. In contrast to the benefits of intra-tumoral expression of CCL5 and CXCL9-11, high levels of CCL22/MDC, the CCR4 ligand preferentially attracting T_{regs}, can be associated with reduced survival, as shown in ovarian cancer patients. Progression of cancer is known to be associated with the paucity of functional CTLs and accumulation of myeloid-derived suppressor cells (MDSCs) and regulatory T cells (T_{regs}), in a CXCL12 and CCL22-dependent mechanism. These considerations suggest that effective immunotherapies of cancer may need to involve vaccination or adoptive transfer of ex vivo-induced CTLs to restore local immune surveillance. We have previously shown that MDSCs are the key source of PGE2 and other suppressive factors in OvCa and that the EP2- and EP4-mediated ability of PGE₂ to enhance COX2 expression leads to positive feedback between PGE₂ and COX2, the key regulator of PGE₂ synthesis, that is required for the maintenance of suppressive functions of MDSCs in OvCa environment. Moreover we have recently reported that the PGE₂-induced SDF1/CXCL12 production and CXCR4 expression may contribute to the local accumulation and retention of MDSCs in OvCa ascites. Similarly, high numbers T_{reg} cells, another type of suppressive immune cells, and low CTL/T_{reg} ratios have also been shown to be negative prognostic factors for cancer patients. The key role of CCL22 in attracting CCR4-expressing T_{regs} to OvCa, the negative prognostic value of CCL22, and our observations that PGE₂ constitutes a key CCL22-inducing factor, suggest that limiting the production of PGE₂ in cancer environment may have therapeutic value by reducing T_{reg} influx. PGE₂ acting via EP2 and EP4 receptors, can also promote the development of T_{regs}. Moreover, we and others have shown that PGE₂ can impair the interaction of DCs with naïve, memory and effector cells, while promoting their interaction with T_{regs}. PGE₂ is also involved in mediating the suppressive activity of T_{regs}.

The level of T cell infiltration of human melanomas, known to be an independent prognostic factor of melanoma survival, shows the strongest correlation with the expression of CXCR3 ligands; CXCL9 and (produced in lesser amounts) CXCL10. The production of CXCL9 by tumor-infiltrating macrophages is particularly effective in primary melanoma lesions, rather than in metastatic tissues, raising the possibility that primary and metastatic tumors may differentially modulate the CK production in the infiltrating APCs. In accordance with the data showing the that T cell-expressed CXCR3 correlates with long term survival, these data suggest that CXCR3 ligands constitute important CKs allowing T cell entry into melanomas, making the induction of CXCR3 on vaccination-induced T cells, and the induction of CXCR3 ligands on non-infiltrated tumor lesions, interesting targets of cancer immunotherapy.

Similar to the clinical studies also mouse studies have shown that most of the antitumor activity is associated with L-selectin^{low}-expressing T cells, and several groups have recently demonstrated the important role of tumor-associated chemokines in tumor rejection. Antitumor activities of adenoviral transfection, or direct injection with chemokines, have been demonstrated in case of CCL3 (MIP1α, ligand for CCR1 and CCR5), CCL7 (MCP3; ligand for CCL1, CCL2, CCL3), CCL16 (LEC; ligand for CCR1), CCL19 (MIP3β; ligand for CCR7) and CXCL11 (ITAC: CXCR3 ligand) in mouse models of adenocarcinoma, P815 mastocytoma, distinct mouse models of breast cancer, and lymphoma. It was shown that XCL1 (lymphotactin; ligand for XCR1)-transduced DCs show superior therapeutic efficacy against B16 melanoma. In SP2/0 myeloma model, it was also shown that the direct transfection of myeloma cells with CXCL1 (lymphotactin; ligand for CXCR1) induces tumor rejection mediated by CXCR1-positive CD4⁺ and CD8⁺ T cells. Keshaw and colleagues demonstrated frequent expression of CXCL1 (Gro-α) by melanomas, and attempted to enhance the effectiveness of cancer immunotherapy by transfecting the receptor for this chemokine (CXCR2) into T cells.

While the attraction of different subsets of T cells to different tumor types is known to be regulated by a complex network of multiple chemokines, our current functional data (see Detailed Description and Figures) indicate that the regimens aimed at the enhancement of tumor-associated CXCR3-ligands and CCR5 ligands uniformly in all tumor lesions can promote the influx of effector CD8⁺ T cells (both spontaneously-arising TILs and induced by cancer vaccines; including αDC1 vaccine that is known to enhance the expression of CCR5 and CXCR3 on tumor specific CTLs CTLs). The known role of CXCR3 and CCR5 in the attraction of Th1 cells and NK cells suggests that such regimens may also be able to promote the entry of these additional types of desirable cells into tumors.

Since not only the spontaneously arising tumor-specific effector cells, but also tumor-specific effector cells induced by different cancer vaccines, including alpha-DC1s or other type of type-1-polarized DCs (such as induced by the combination of LPS and IFNγ or by the combination of TNFα and IFNγ or TNFα and IL-1β and IFNγ), express high levels of CCR5 or CXCR3 on tumor-specific T cells, the induction of the CCR5 ligands or/and CXCR3 ligands in tumor tissues may be particularly effective in combination with the application of such vaccines.

While the differences in homing properties of different T cell subsets have been known for over 15 years, a series of recent studies demonstrated the key role DCs in this respect. DCs isolated from Peyers' Patches or treated with retinoids, show the ability to induce gut-homing properties in naïve T cells. Similarly, migratory APCs have been recently demonstrated as responsible for the imprinting of the ability of T cells to home to the central nervous system. In support of the notion that the migratory capacity of human melanoma-specific T cells can be affected by DC-related factors (delivery of “Signal 4”), it was shown that enhanced expression of functional CLA (cutaneous homing receptor; ligand for skin endothelium-expressed ELAM) and enhanced migration of effector CTLs to metastatic melanomas lesions in the skin can be induced by the treatment of patients with systemic IL-12. Indeed Ogg and colleagues showed that high expression of CLA on CTLs from vitiligo patients allows these cells to efficiently enter skin and initiate melanocyte destruction. Berger and colleagues have recently reported that vaccination with monocyte-derived DCs can induce melanoma-specific T cells that home to both the skin and to visceral metastases.

However, the possibility of enhancing the efficacy of cancer immunotherapy involving adoptive transfer of T cells by modulating the pattern of chemokines at tumor sites, to facilitate the tumor entry of the effector-type T cells has not been explored. Similar, so far, no study has yet addressed the possibility of enhancing the efficacy of cancer immunotherapy by modulating the pattern of chemokines at tumor sites, to facilitate the tumor entry of the effector-type T cells induced by vaccines or arising spontaneously in the patients.

Hyper-activation of NF-κB, a common feature of many types of cancer tissues. NF-κB signaling, commonly triggered by ligands of multiple Toll-like receptors (TLRs; including TLR3 a receptor for double-stranded RNA); and multiple other proinflammatory stimuli, including TNFα or IL1β, is an important requirement for the induction of both T_{reg}- and T_{eff}-attracting classes of chemokines. However, so far it has not been known how to utilize the pattern of NF-κB signaling and to modulate it in order to selectively or at least preferentially enhance the production of T_{eff}-attracting chemokines in tumor tissues, rather than marginal tissues, in order to selectively direct T_{eff} cells to tumors.

Several studies have previously demonstrated the ability of IFNs and TLR ligands to promote the production of T_{eff}-recruiting chemokines, including CCR5- and CXCR3 ligands. On the other hand, PGE₂, a factor overproduced by CRC and other tumors and associated with negative prognosis, has been previously implicated in the suppression of such T_{eff}-attracting CKs, and promoting Th2/Treg-recruiting CKs. However, it remains unknown whether these different groups of inflammatory factors affect the balance of production of Teff v Treg-attracting CKs in the primary and metastatic cancer tissue, whether any of these factors show any synergistic activities, and whether it is possible to apply any combinations of the CK-modulating factors to selectively enhance the production of the Teff-attracting CKs (without enhancing the production of Treg-attracting CKs) and how to assure the selectivity of such effects to tumor tissues (rather than healthy tissues), to focus the desirable types of immune cells on the disease sites.

Such possibility has been provided by our current data that demonstrate that tumor microenvironments not only spontaneously hyper-activate NF-κB, but also respond to the proposed treatments with further-enhanced levels of NF-κB activation. Since NF-κB activation, critically involved in tumor survival and growth, represents an intrinsic feature of many tumor types, the current data suggest that the currently-described NF-κB-targeting modulation of the tumor microenvironment may be applicable to multiple types of cancer.

In contrast to the cancer settings, where a preferential mobilization of the effector immune cells is believed to be beneficial, in the settings of chronic inflammation, autoimmune diseases, transplant rejection or graft versus host disease following bone marrow transplantation it is believed that the effector immune cells are the cause of the pathology, while the suppressive cells, such as Tregs and MDSCs are beneficial. In such cases, the selective suppression of the effector cell-attracting chemokines and the enhancement of the Treg- and MDSC-attracting chemokines is likely to result in the therapeutic benefit.

### SUMMARY

Therapies effective for the treatment and prevention of cancer and other diseases are disclosed herein. These methods include the administration of a therapeutically effective amount of at least two different agents that act synergistically to differentially modulate the production of IP-10 (CXCL10), and RANTES (CCL5) in tumor tissues (or other disease-effected tissues), versus (an opposite effect or no change) the production of CCL22, the chemokine known to attract undesirable regulatory T cells.

Our novel and unexpected data demonstrate that the patterns of the NF-κB-driven production of T_{reg}-versus T_{eff}-attracting classes of chemokines can be differentially regulated by targeting IFNs and prostaglandin synthesis. Both prostaglandins and IFNs are known to regulate the production of chemokines, but their interplay with TLR ligands and other activators of NF-κB pathways in the reciprocal regulation of the T_{reg}-versus T_{eff}-attracting classes of chemokines has not been used for therapeutic purposes. Since several studies have indicated the propensity of multiple types of tumors to over-express COX2 and over-produce a COX2 product PGE₂. It is likely that the proposed strategies to selectively enhance the production of T_{eff}-attracting chemokines in tumor tissues, rather than marginal tissues, in order to selectively direct T_{eff} cells to tumors and applicable to multiple types of tumors and other diseases associated with NF-κB imbalance, such as infections, chronic inflammations, premalignant states, autoimmune phenomena, or transplant rejection, including the rejection of transplanted organs, tissues, and isolated cells, including transplant rejection and graft-versus-host (GvH) disease.

In case of prevention or treatment of cancer, some premalignant states and many infections, our data suggest a benefit of a combined application of TLR ligands or other activators of NF-κB pathways with the inhibitors of prostanoids (and potentially with the inhibitors of prostaglandin receptors and/or the inhibitors of the production of other cAMP-elevating agents or the inhibitors of cAMP signaling) and with prior, concomitant or subsequent administration of IFNα and/or other type I and type II interferons), in order to selectively enhance the production of the T_{eff}-attracting chemokines while suppressing the production of T_{reg}-attracting chemokines.

On the other hand, in case of prevention or treatment of the states associated with the undesirable over-activation of the immune system (chronic inflammations, some premalignant states, autoimmune phenomena, or transplant rejection, including the rejection of transplanted organs, tissues, and isolated cells, including transplant rejection and graft-versus-host (GvH) disease), our data suggest a benefit of combined application of TLR ligands or other activators of NF-κB pathways with prostanoids or other cAMP-elevating agents (and with potential inhibitors of IFN production of IFN responsiveness), in order to selectively enhance the production of the T_{reg}-attracting chemokines while suppressing the production of T_{eff}-attracting chemokines.

In some embodiments, methods are provided for treating or preventing the incidence or recurrence of colorectal cancer, melanoma, non-melanoma skin cancers, glioma, ovarian cancer, breast cancer, lung cancer, endometrial cancer, cervical cancer, gastric cancer, esophageal cancer, pancreatic cancer, biliary cancer, renal cancer, bladder cancer, vulvar cancer, neuroendocrine cancer, prostate cancer, head and neck cancer, soft-tissue sarcoma, bone cancer, mesothelioma, cancer of endothelial origin, hematologic malignancy including but not limited to multiple myeloma, lymphomas and leukemias, a pre-malignant lesion known to be associated with increased risk of developing cancer or other forms of cancer in a subject. These methods include administering to the subject at therapeutically effective amount of at least two different agents, such as, but not limited to interferons, inhibitors of prostanoids synthesis and a Toll-like receptor (TLR) agonist.

In some embodiments, methods are provided for treating cancer or preventing cancer's occurrence or recurrence in a subject include administering to the subject at therapeutically effective amount of a prostaglandin inhibitor (such as an inhibitor of prostaglandin synthesis or prostaglandin responsiveness) or other cAMP suppressing agent that increases IP-10/CXCL10 production and inhibits MDC/CCL22 production and a therapeutically effective amount of a Toll-like receptor (TLR) agonist.

In other embodiments, methods are provided for treating cancer or preventing cancer's occurrence or recurrence in a subject by administering to the subject a therapeutically effective amount of an interferon or an agent that increases IP-10 activity and a therapeutically effective amount of a prostaglandin synthesis inhibitor, thereby treating or preventing colorectal cancer in the subject.

Since not only the spontaneously arising tumor-specific effector cells, but also tumor-specific effector cells induced by different cancer vaccines, including alpha-DC1s or other type of type-1-polarized DCs (such as induced by the combination of LPS and IFNγ or by the combination of TNFα and IFNγ), express high levels of CCR5 or CXCR3 on tumor-specific T cells, the tumor-selective induction of CCR5 ligands or/and CXCR3 ligands in tumor tissues may be particularly effective in combination with the application of such vaccines.

The foregoing and other features and advantages will become more apparent from the following detailed description of several embodiments, which proceeds with reference to the accompanying figures.

### BRIEF DESCRIPTION OF DRAWINGS

FIG. 1. Superior activity of type-1-polarized dendritic cells (aDCs) in inducing functional CTLs expressing CXCR3 and CCR5. A-C: αDC1 are superior inducers of CD8+ T cell responses against multiple tumor-associated epitopes. (A) αDC1 or sDC were loaded with HLA-A2-presented melanoma-associated CTL epitopes (MART-1, gp100 & tyrosinase) and used to sensitize autologous CD8+ T cells from HLA-A2+melanoma patient (stage IV) in an in vitro sensitization (IVS) system. After one additional round of stimulation with PBMCs (to test stability of differences) CD8+ T cells responsive to individual peptides were detected by ELISPOT. (B) αDC1-primed CTLs kill HLA-A2-matched melanoma cells. The ability of differentially-sensitized CD8+ T cells to kill HLA-A2+ melanoma cell line was tested at day 20, using chromium release assay. (C) αDC1 are superior inducers of CD8+ T cell responses against CEA (CAP-1 epitope). IVS with blood of a colorectal cancer patient. (D) αDC1 are superior inducers of CXCR3 and CCR5 on melanoma-specific CD8+ T cells in the IVS system (described in panel A). Chemokine receptor expression on MART-1-Tetramer⁺ CD8⁺ T cells was analyzed by flow cytometry. (D) High CTL-inducing activity of αDC1 in IVS system, using αDC1 or sDC loaded with autologous CLL cells. Two rounds of DC-stimulation and no PBMC stimulation was used. (E) Differential CCR5 expression and CCL5 responsiveness on αDC1 and standard (s)DC-primed CD8+ T cells in the IVS system using the SEB-driven model of T cell activation.

FIG. 2. Heterogeneous expression of T_{eff}- or T_{reg}-attracting chemokines in different tumors. Tumor biopsies from colon cancer or melanoma patients were lysed, RNA extracted and Taqman analysis was performed. (A) Heterogeneous expression of T_{reg}- and T_{eff}-attracting chemokines in skin-, lymph node- and skin lesions of melanoma. (B) Heterogeneous expression of CXCL10/IP10 and CCL22 in different colorectal cancer lesions.

FIG. 3. Presence of T_{eff}- and T_{reg} markers in tumors correlates with intra-tumoral expression of, respectively, T_{eff}- or T_{reg}-attracting chemokines. Tumor biopsies from colon cancer patients were lysed, RNA extracted and Taqman analysis of various markers was performed. (A) Correlation between T_{eff} markers (CD8 and Granzyme B; GZMB) and T_{eff}-attracting chemokines (CCL5 and CXCL10) in tumor lesions. (B) Correlation between T_{reg} markers (FOXP3 and GITR) and the chemokine CCL22 in tumor lesions.

FIG. 4. Presence of T_{reg} and T_{eff} markers in tumors correlate with intra-tumoral expression of T_{eff}- and T_{reg}-attracting chemokines. (A) Expression of an alternative CXCR3 ligand, CXCL9, is correlated with local expression of CXCL10 and with T_{eff} markers, CD8 and GZMB. (B) Correlation between CCL22 and COX-2 (C) Example: Lack of correlation between CCL22 and CXCL13.

FIG. 5. Interplay between COX inhibitor indomethacin, IFNα and poly-I:C in the induction of a desirable patterns of chemokine expression in isolated cell cultures. (A) Dose-dependent impact of IFNa and poly-I:C on the production of T_{eff-}, and T_{reg}-attracting chemokines by in vitro generated macrophages (see M&M) and in fibroblasts (obtained from Cascade Biological). Data from one representative experiment of three. (B) Effects of indomethacin on T_{eff-}, and T_{reg}-attracting chemokines produced by macrophages (N=3) by ELISA analysis. The concentrations of chemokines in 48 hr. cultures were analyzed by ELISA. Note the suppression of CCL22 production by indomethacin.

FIG. 6. Heterogeneous response pattern of different tumor tissues to individual chemokine modulators and their uniform response to the combination of IFNα, poly-I:C and indomethacin. (A) Fresh tumor samples from 11 patients with metastatic colorectal cancer were untreated or treated with IFNα and poly-I:C either individually or in combination for 48 hours. The release of CCL5 and CXCL10 into culture media was analyzed by ELISA. Numbers indicate the prevalence of tumors with each chemokine pattern (respective patterns A, B or C). (B) ELISA analysis of CCL5, CCL22 and CXCL10 in tumors untreated or treated with IFNα+pI:C, with or without indomethacin. (C) Heterogeneous response of different tumor lesions from the same patient (CCL5 and CXCL10 production) to the individual components of the chemokine-modulating cocktail. (D) Indomethacin and celecoxib enhance the IFNα/poly-I:C-induced T_{eff}-attracting chemokine expression, but suppress T_{reg-}attracting chemokine expression in colorectal cancer lesions. All cultures were for 48 hr. Combined data from the tumors of 3 different patients (n=3).

FIG. 7. Combination of IFNα, poly-I:C and indomethacin, consistently up regulates T_{eff}-attracting chemokines and consistently suppresses T_{reg}-attracting chemokines in tumor tissues. (A) In-situ hybridization for respective chemokine mRNA (black grains) in tumor biopsies which were either left untreated or treated with the combination of indomethacin, IFNa and poly-I:C (IAP). (B) ELISA analysis of the chemokine contents in the supernatants of 48 hour-cultured tumor tissues (untreated or treated) from 10 different patients.

FIG. 8. NF-κB-dependent selective enhancement of CXCL10 production in different tumor tissues following exposure to the combination of IFNα, poly-I:C and indomethacin. (A) ELISA for CXCL10 expression in matched normal liver and liver metastatic tissues from 10 different patients either untreated (left panel) or treated (right panel). (B) Average number of cells counted per field (confocal microscopy; in a total of 10 fields) showing nuclear translocation of NF-κB in normal liver or liver metastatic tissues either untreated or treated (right panel). Representative images of each condition are shown in the left panel. (C) ELISA analysis of CXCL10 production by the matched normal liver and liver-metastatic colorectal cancer tissues, either untreated or treated (IFNα, poly-I:C and indomethacin), in the absence or presence of 20 μM CAY10470 (NF-κB inhibitor). D) Selectivity of chemokine modulation in melanoma lesions (versus healthy skin). Different melanoma lesions or marginal healthy skin were treated with IFNα+Poly-I:C and the secretion levels of CXCL10/IP10 were measured by ELISA

FIG. 9. Selective induction of CXCL10 and CCL5 in liver metastases compared to normal liver tissues: role of NF-κB. Matched samples of marginal liver tissues and liver-metastatic colorectal cancer tissues (3 biopsies in 1 ml in 24 well plate), were cultured for 24 hrs either untreated or treated with IFNα+poly-I:C+indomethacin and (A) analyzed for CCL5 and CXCL10 expression by Taqman. (B) Tissues were untreated or treated with IFNα+poly-I:C+indomethacin in absence or presence of 20 μM CAY10470 (NF-κB inhibitor). The supernatants were analyzed for CCL5 production by ELISA (see matched CXCL10 data in FIG. 4). (C) CAY10470 (20 μM) effects on liver glycogen phosphorylase mRNA expression in matched marginal liver tissue and liver-metastatic colorectal cancer.

FIG. 10. IFNα, poly-I:C and indomethacin-treated tumors show enhanced ability to attract T_{eff}; but strongly-reduced ability to attract T_{regs}. (A) Ex vivo generated T_{eff} (left) or isolated CD8⁺ tumor-infiltrating lymphocytes (right) (see Materials and Methods) were allowed to migrate towards supernatants from either untreated or treated tumors from 3 different patients in transwell chemotaxis assays. (B) Negatively-isolated total CD4⁺ T cells were allowed to migrate towards the treated- or untreated tumor supernatants. Migrating cells were lysed and analyzed for FOXP3 expression by Taqman. U.D.: undetectable.

FIG. 11. Modulation of the TLR sensors by IFN. Whole tumor tissue samples, blood-isolated NK cells, or monocyte-derived DCs, were analyzed for the spontaneous, and IFNα-(or PGE2)-inducible expression of TLR3 and MyD88 mRNA (Taqman).

FIG. 12. Different TLR ligands and alternative NF-κB activators synergize with type I and type II interferons in the induction of IP10/CXCL10. Isolated cells were treated with IFNα or IFNγ alone or in combination with poly-I:C (TLR3 ligand), LPS (TLR4 ligand) or a pro-inflammatory cytokine, TNFα. (B) The induction of IP10/CXCL10 gete was measured by Taqman.

FIG. 13. Combination of celecoxib (COX2 inhibitor), IFNα and poly-I:C, counteract the undesirable elevation of the ratio between Treg-attracting and Teff-attracting chemokines (ratio between CCL22/CXCL10) in melanoma tissue treated by a chemotherapeutic agent, melphalan. Tumor biopsies were cultured ex vivo in the presence of increasing concentrations of melphalan in the absence or presence of the triple combination (CAP) of the Celecoxib, IFNα and poly-I:C. Secretion levels of CC22 and CXCL10 were measured by ELISAs.

### DETAILED DESCRIPTION

Therapies effective for the treatment and prevention of cancer and other diseases are disclosed herein. These methods include the administration of a therapeutically effective amount of agents that increase the local production of effector cell-attracting chemokines within tumor lesions, with concomitant suppression of local production of undesirable chemokines that attract regulatory T(reg) cells. These methods include administering to the subject at therapeutically effective amount of a Toll-like receptor (TLR) agonist in combination with a blocker of prostaglandin synthesis, in combination with a type-1 interferon, or in combination with both a blocker of prostaglandin synthesis and with a type-1 interferon, thereby treating or preventing cancer or an infectious disease or preventing the recurrence of such in the subject. Alternatively, the methods derived from the same paradigms, but meant to treat or prevent autoimmune disease, chronic inflammatory disease, or transplant rejection include the combination of a Toll-like receptor (TLR) agonist in combination with a prostaglandin, including, but not limited to prostaglandin E2 (PGE2), or other activator of the adelylate cyclase/cAMP/CREB signaling pathway.

### I. GENERAL TERMS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Chemokines: Immune chemoattractants inducing the migration of immune cells towards its source (against the gradient). IP10/CXCL10, RANTES/CCL5, and MDC/CCL22 are examples of chemokines. IP10 (together with two related chemokines MIG/CXCL9 and I-TAC/CXCL11) all bind to the same chemokine receptor: CXCR3, mainly expressed on effector-type-immune cells, such as Th1 cells, BK cells and CTLs, that are all known to promote tumor rejection. RANTES/CCL5 binds to CCR5, CCR3 and CCR1. Similar to the CXCR3 ligands it is also known to mainly attract immune effector cells and promote tumor rejection. MDC/CCL22 binds to CCL4, expressed mainly on regulatory T(reg) cells, the cell type that is known to be involved to protect tumors from immune destruction and promotes tumor progression. In contrast to the “desirable chemokines IP10, MIG, ITAC and RANTES, the “undesirable” chemokines include MDC/CCL22 that mediates attraction of undesirable Treg cells and SDF1/CXCL12 that mediates the attraction of undesirable Treg cells and MDSCs.

Chemotherapeutic agents: Any chemical agent with therapeutic usefulness in the treatment of diseases characterized by abnormal cell growth. Such diseases include tumors, neoplasms, and cancer. In one embodiment, a chemotherapeutic agent is an agent of use in treating a colorectal cancer, melanoma or another tumor. In one embodiment, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., ® 2000 Churchill Livingstone, Inc.; Baltzer, L., Berkery, R. (eds.): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993).

Combination chemotherapy is the administration of more than one agent to treat cancer. One example is the administration of an antibody or a fragment thereof used in combination with a radioactive or chemical compound.

Cyclooxygenase Inhibitor:

A compound that selectively inhibits the cyclooxygenase-2 enzyme and/or cyclooxygenase-1 enzyme, reducing the production of prostanoids.

Cyclooxygenase-2 (COX-2) Selective Inhibitor:

A compound that selectively inhibits the cyclooxygenase-2 enzyme over the cyclooxygenase-1 enzyme. In one embodiment, the compound has a cyclooxygenase-2 IC₅₀ of less than about 2 μM and a cyclooxygenase-1 IC₅₀ of greater than about 5 μM, in the human whole blood COX-2 assay (as described in Brideau et al., Inflamm Res., 45: 68-74 (1996)) and also has a selectivity ratio of cyclooxygenase-2 inhibition over cyclooxygenase-1 inhibition of at least about 10, such as at least about 40. In another embodiment, the compound has a cyclooxygenase-1 IC₅₀ of greater than about 1 μM, and preferably of greater than 20 μM. The compound can also inhibit the enzyme, lipoxygenase. Such selectivity may indicate an ability to reduce the incidence of common NSAID-induced side effects.

Interferon-Inducible Protein-10 (IP-10):

A cytokine also known as immune protein-10; 10 kDa interferon-inducible protein] also: gamma-IP-10, INP-10, or C7. The rat homolog of this protein is called mob-1. The name CXCL10 has also been proposed for this factor. The gene symbol is SCYB 10. Based on the presence of a conserved three-dimensional motif and direct microbicidal activity, IP-10 has been classified as a kinocidin (microbicidal chemokine).

In humans, the native protein has a length of 98 amino acids. It has homology to PF4 (platelet factor-4) and belongs to the family of chemotactic cytokines known as Chemokines. IP-10 is also related to a gene called CRG-2 (see: CRG, cytokine responsive genes). Murine CRG-2 and human IP-10 are considered homologous. The human IP-10 genes contains four exons and maps to chromosome 4q12-21 in the vicinity of other genes encoding chemokines.

The receptor for IP-10 is CXCR3. IP-10 has been shown to bind to the virus-encoded viroceptor M3. The expression of IP-10 from a variety of cells, including monocytes, endothelial cells, keratinocytes, and fibroblasts, is induced by IFN-gamma and TNF-alpha. Human neutrophils produce IP-10 in response to IFN-gamma in combination with either TNF-alpha or bacterial lipopolysaccharides.

Neoplasia, Malignancy, Cancer or Tumor:

The result of abnormal and uncontrolled growth of cells. Neoplasia, malignancy, cancer and tumor are often used interchangeably. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, melanoma basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Preventing, Treating or Ameliorating a Disease:

“Preventing” a disease refers to inhibiting the full development of a disease, or delaying the development of the disease. “Treating” refers to a therapeutic intervention that ameliorates a sign or symptom of a disease or pathological condition after it has begun to develop. “Ameliorating” refers to the reduction in the number or severity of signs or symptoms of a disease, such as cancer.

PGE₂ Production.

The process of PGE₂ synthesis involves phospholipase A2 (PLA2) family members, that mobilize arachidonic acid from cellular membranes, cyclooxygenases (constitutively-active COX1 and inducible COX2) that convert arachidonic acid into prostaglandin H₂ (PGH₂), and prostaglandin E synthase (PGES), needed for the final formulation of PGE₂. While the rate of PGE₂ synthesis and the resulting inflammatory process can be affected by additional factors, such as local availability of AA, in most physiologic conditions, the rate of PGE₂ synthesis is controlled by local expression and activity of COX2.

PGE₂ Degradation.

The rate of PGE₂ degradation is controlled by 15-hydroxyprostaglandin dehydrogenase (15-PGDH), suggesting that in addition to the rate of PGE₂ synthesis, also the rate of PGE₂ decay constitutes a target for immunomodulation.

PGE₂ Responsiveness.

Four different PGE₂ receptors are EP1, EP2, EP3 and EP4. The signaling through the two Gₛ-coupled receptors, EP2 and EP4, is mediated by the adenylate cyclase-triggered cAMP/PKA/CREB pathway, mediating the dominant aspects of the anti-inflammatory and suppressive activity of PGE₂. While EP2 is believed to signal in a largely cAMP-dependent fashion, EP4 also activates the PI3K-dependent ERK1/2 pathway. However, both EP2 and EP4 have been shown to activate the GSK3/β-catenin pathway.

The expression of EP2 and the resulting responsiveness to PGE₂ can be suppressed by hyper-methylation, as observed in patients with idiopathic lung fibrosis. These observations raise the possibility that, in addition to the regulation of PGE₂ production and its degradation, the regulation of PGE₂ responsiveness at the level of expression of individual PGE₂ receptors can also contribute to the pathogenesis of human disease and be exploited in their therapy. In support of this possibility, the use of synthetic inhibitors, preferentially affecting EP2, EP3, or EP4 signaling, allow for differential suppression of different aspects of PGE₂ activity (reviewed in).

Prostaglandin (PG) Synthesis Inhibitors:

Factor which inhibit the synthesis of PGs in general or the synthesis of a specific type of PGs. PG synthesis inhibitors include nonselective inhibitors of COX-1 and COX-2, the two key enzymes in the PG synthesis pathway, and selective inhibitors of COX-2, which are believed to be more specific to COX-2 and less toxic. The examples of non-selective PG inhibitors include aspirin, indomethacin, or ibuprofen (Advil, Motrin). The examples of COX-2-selective inhibitors include Celecoxib (Celebrex) and rofecoxib (Vioxx). The example of COX-1-specific inhibitor is sulindac (Clinoril). Other drugs that suppress prostaglandin synthesis include steroids (example: hydrocortisone, cortisol, prednisone, or dexamethasone) and acetaminophen (Tylenol, Panadol), commonly used as anti-inflammatory, antipyretic and analgesic drugs. Examples of the most commonly used selective COX2 inhibitors include celecoxib, alecoxib, valdecoxib, and rofecoxib.

Examples of the most commonly used non-selective COX 1 and COX2 inhibitors include: acetylsalicylic acid (aspirin) and other salicylates, acetaminophen (Tylenol), ibuprofen (Advil, Motrin, Nuprin, Rufen), naproxen (Naprosyn, Aleve), nabumetone (Relafen), or diclofenac (Cataflam).

Prostaglandin (PG) Signaling Inhibitors:

Prostaglandins signal through numerous receptors, with the key immunosuppressive effects being mediated by the activation of adenylate cyclase, the resulting elevation of the intracellular cyclic (c)AMP, PKA and the downstream activation of the PKA/CREB pathway.

Another level of interference with the PG responsiveness includes the interference with their binging to PG receptors. In case of PGE2, the two key cAMP-activating receptors are EP2 and EP4, for which a number of specific inhibitors exist.

The increase of cAMP levels induced by prostaglandins or other factors can be prevented by phosphodiesterases (PDEs; currently known 6 types, PDE1-PDE5 and PDE10, which reduce the levels of intracellular cAMP). PDEs can be controlled by phosphodiesterase inhibitors, which include such substances as xanthines (caffeine, aminophylline, IBMX, pentoxyphylline, theobromine, theophylline, or paraxanthine), which all increase the levels of intracellular cAMP, and the more selective synthetic and natural factors, including vinpocetine, cilostazol, inaminone, cilostazol, mesembrine, rolipram, ibudilast, drotaverine, piclamilast, sildafenil, tadalafil, verdenafil, or papaverine.

Furthermore, interference with PGE2 signaling (or with the signaling of other cAMP-elevating factors, such as histamine, of beta-adrenergic agonists) can be achieved by the inhibition of downstream signals of cAMP, such as PKA or CREB.

Therapeutically Effective Amount:

An amount of a therapeutic agent (such as IP-10 or an agent that increases the production of IP-10/CXCL10 and/or RANTES/CCL5, decreases the production of CCL22, or increases the numbers or the quality of the immune cells expressing CXCR3 and/or CCR5 and capable of migrating towards CXCL10 or CCL5) that alone, or together with one or more additional therapeutic agents, induces the desired response, such as decreasing the risk of developing cancer or decreasing the signs and symptoms of cancer. In one example, it is an amount of an agent needed to prevent or delay the development of a tumor, such as melanoma or colorectal cancer, in a subject. In another example, it is an amount of the agent needed to prevent or delay the metastasis of a tumor, cause regression of an existing tumor, or treat one or more signs or symptoms associated with a tumor in a subject, such as a subject having melanoma or colorectal cancer. Ideally, a therapeutically effective amount provides a therapeutic effect without causing substantial adverse effects in the subject. The preparations disclosed herein are administered in therapeutically effective amounts.

In one example, a desired response is to prevent the development of a tumor. In another example, a desired response is to delay the development, progression, or metastasis of a tumor, for example, by at least about 3 months, at least about six months, at least about one year, at least about two years, at least about five years, or at least about ten years. In a further example, a desired response is to decrease the occurrence of cancer, such as colorectal cancer or melanoma. In another example, a desired response is to decrease the signs and symptoms of cancer, such as the size, volume, or number of tumors or metastases. For example, the composition can in some examples decrease the size, volume, or number of tumors (such as colorectal tumors) by a desired amount, for example by at least 5%, at least 10%, at least 15%, at least 20%, at least 25%, at least 30%, at least 50%, at least 75%, or even at least 90%, as compared to a response in the absence of the therapeutic composition.

In general, an effective amount of a composition administered to a human subject will vary depending upon a number of factors associated with that subject, for example the overall health of the subject, the condition to be treated, or the severity of the condition. An effective amount of a composition can be determined by varying the dosage of the product and measuring the resulting therapeutic response, such as the decrease in occurrence of cancer, such as colorectal cancer or melanoma, or the decrease in the size, volume or number of tumors. Any agent can be administered in a single dose, or in several doses, as needed to obtain the desired response. However, the effective amount can be dependent on the source applied, the subject being treated, the severity and type of the condition being treated, and the manner of administration, including the route, rate, and frequency of administration, as well as the formulation of each of the active compounds or their combination, as well as the relative timing of the administration of each of the components in relation to each other and to other elements of the overall patient care (surgery, radiotherapy, chemotherapy, biologic therapy or any means of symptom management).

Toll-Like Receptor (TLR):

A family of receptors which plays a fundamental role in pathogen recognition and activation of innate immunity. TLRs are highly conserved from Drosophila to humans and share structural and functional similarities. They recognize pathogen-associated molecular patterns (PAMPs) that are expressed on infectious agents, and mediate the production of cytokines necessary for the development of effective immunity. There are a total of total of 13 mammalian TLRs, including nine (TLR1-9) that have been extensively studied and are known to activate the NF-κB pathway.

Toll-Like Receptor 3 (TLR3):

A member of the Toll-like receptor (TLR) family. Its amino acid sequence of is shown in NCBI accession number NP_{—}003256, as of Jan. 2, 2009, the disclosure of which is incorporated herein by reference. TLR3 is a member of the Toll-like receptor (TLR). This receptor is most abundantly expressed in placenta and pancreas, and is restricted to the dendritic subpopulation of the leukocytes. It recognizes dsRNA associated with viral infection, and induces the activation of NF-κB and the production of type I interferons. The TLR3 mRNA sequence is described in NCBI accession number NM_{—}003265, which is also incorporated by reference as of Jan. 2, 2009. TLR3 is described in WO 98/50547, the disclosure of which is also incorporated herein by reference). The term “TLR3 gene” designates the Toll Like Receptor 3 gene, as well as variants, analogs and fragments thereof, including alleles thereof (e.g., germline mutations). Such variants include, for instance, naturally-occurring variants due to allelic variations between individuals (e.g., polymorphisms), alternative splicing forms, etc. In several embodiments, variants are substantially homologous to the NCBI accession No. NM_{—}003265 sequence, such as have a nucleotide sequence identity of at least about 65%, at least about 75%, at least about 85%, or at least about 95% with the reference sequence. Variants and analogs of a TLR3 gene also include nucleic acid sequences, which hybridize to a sequence as defined above (or a complementary strand thereof) under highly stringent hybridization conditions. Genetic polymorphisms of the human TLR3DNA sequence are known, for example allelic variations in the cytoplasmic region of TLR3 gene and in the immediate 5′ sequence of the TLR3 gene (see Piriel et al. (2005) Tissue Antigens 66(2): 125, the disclosure of which is incorporated herein by reference), for example the C/T polymorphism at position 2593, the C/A polymorphism at position 2642 and the AJG polymorphism at position 2690 in the TLR3 gene.

### II. AGENTS THAT INCREASE THE PRODUCTION OF CXCR3LIGANDS AND CCR5LIGANDS, SUCH AS TYPE I INTERFERONS (IFN-α AND IFN-β) AND TYPE-II INTERFERONS (IFN-γ)

The term “interferon-alpha” as used herein refers to a family of related polypeptides that inhibit viral replication and cellular proliferation and modulate immune response. The term “IFN-α” “IFN-alpha” or “IFN-a” includes IFN-a polypeptides that are naturally occurring; non-naturally-occurring IFN-a polypeptides; and analogs of naturally occurring or non-naturally occurring IFN-a that retain antiviral activity of a parent naturally-occurring or non-naturally occurring IFN-a.

Suitable alpha interferons include, but are not limited to, naturally-occurring IFN-a (including, but not limited to, naturally occurring IFN-a2a, IFN-a2b); recombinant interferon alpha-2b such as Intron A, interferon available from Schering Corporation, Kenilworth, N.J.; recombinant interferon alpha-2a such as Roferon (g) interferon available from Hoffmann-La Roche, Nutley, N.J.; recombinant interferon alpha-2C such as Berofor alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn.

IFN-alpha also encompasses consensus IFN-a. “Consensus IFN-a” refers to a non-naturally-occurring polypeptide, which includes those amino acid residues that are common to all naturally-occurring human leukocyte IFN-a subtype sequences and which includes, at one or more of those positions where there is no amino acid common to all subtypes, an amino acid which predominantly occurs at that position, provided that at any such position where there is no amino acid common to all subtypes, the polypeptide excludes any amino acid residue which is not present in at least one naturally-occurring subtype Amino acid residues that are common to all naturally-occurring human leukocyte IFN-a subtype sequences (“common amino acid residues”), and amino acid residues that occur predominantly at non-common residues (“consensus amino acid residues”) are known in the art. Consensus IFN-a (also referred to as “CIFN” and “IFN-con” and “consensus interferon”) encompasses but is not limited to the amino acid sequences designated IFN-con1, IFN-con2 and IFN-con3 which are disclosed in U.S. Pat. Nos. 4,695,623 and 4,897,471; and consensus interferon as defined by determination of a consensus sequence of naturally occurring interferon alphas (e.g., Infergen (g), InterMune, Inc., Brisbane, Calif.). IFN-con1 is the consensus interferon agent in the Infergen (D alfacon-1 product). The Infergen consensus interferon product is referred to herein by its brand name (Infergen (Z) or by its generic name (interferon alfacon-1)). DNA sequences encoding IFN-con may be synthesized as described in the aforementioned patents or other standard methods. Use of CIFN is of particular interest.

Also suitable for use in the methods disclosed herein are fusion polypeptides comprising an IFN-a and a heterologous polypeptide. Suitable IFN-a fusion polypeptides include, but are not limited to, Albuferon-alpha (a fusion product of human albumin and IFN-a; Human Genome Sciences; see, for example, Osborn et al. (2002) J. Pharmacol. Exp. Therap. 303: 540-548). Also suitable for use in the present invention are gene-shuffled forms of IFN-a. See, for example, Masci et al. (2003) Curr. Oncol. Rep. 5: 108-113.

IFN-a polypeptides can be produced by any known method. DNA sequences encoding IFN-con may be synthesized as described in the above-mentioned patents or other standard methods. In many embodiments, IFN-a polypeptides are the products of expression of manufactured DNA sequences transformed or transfected into bacterial hosts, such as E. coli, or in eukaryotic host cells (for example, yeast; mammalian cells, such as CHO cells; and the like). In these embodiments, the IFN-a is recombinant. Where the host cell is a bacterial host cell, the IFN-α is modified to comprise an N-terminal methionine. IFN-a produced in E. coli is generally purified by procedures known to those skilled in the art and generally described in Klein et al. ((1988) J. Chromatog. 454: 205-215) for IFN-con1.

Bacterially produced IFN-a may comprise a mixture of isoforms with respect to the N-terminal amino acid residue. For example, purified IFN-con may comprise a mixture of isoforms with respect to the N-terminal methionine status. For example, in some embodiments, an IFN-con comprises a mixture of N-terminal methionyl IFN-con, des-methionyl IFN-con with an unblocked N-terminus, and des-methionyl IFN-con with a blocked N-terminus. As one non-limiting example, purified IFN-con1 comprises a mixture of methionyl IFN-con1 des-methionyl IFN-con1 and des-methionyl IFN-con1 with a blocked N-terminus Klein et al. ((1990) Arch. Biochemistry &amp; Biophys. 276: 531-537). Alternatively, IFN-con may comprise a specific, isolated isoform. Isoforms of IFN-con are separated from each other by techniques such as isoelectric focusing which are known to those skilled in the art. IFN-a as described herein may comprise one or more modified amino acid residues, for example, glycosylations, chemical modifications, and the like.

The term “IFN-a” also encompasses derivatives of IFN-a that are derivatized (e.g., are chemically modified) to alter certain properties such as serum half-life. As such, the term “IFN-a” includes glycosylated IFN-a; IFN-a derivatized with polyethylene glycol (“PEGylated IFN-a”); and the like. PEGylated IFN-a, and methods for making this molecule, are discussed U.S. Pat. Nos. 5,382,657; 5,981,709; and 5,951, 974. PEGylated IFN-a encompasses conjugates of PEG and any of the above-described IFN-a molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen&commat;, InterMune, Inc., Brisbane, Calif.).

Any of the above-mentioned IFN-a polypeptides can be modified with one or more polyethylene glycol moieties, for example, PEGylated. The PEG molecule of a PEGylated IFN-a polypeptide is conjugated to one or more amino acid side chains of the IFN-a polypeptide. In some embodiments, the PEGylated IFN-a contains a PEG moiety on only one amino acid. In other embodiments, the PEGylated IFN-a contains a PEG moiety on two or more amino acids, for example the IFN-a contains a PEG moiety attached to two, three, four, five, six, seven, eight, nine, or ten different amino acid residues.

IFN-a may be coupled directly to PEG (such as without a linking group) through an amino group, a sulfhydryl group, a hydroxyl group, or a carboxyl group. In some embodiments, the PEGylated IFN-α is PEGylated at or near the amino terminus (N-terminus) of the IFN-a polypeptide, for example the PEG moiety is conjugated to the IFN-a polypeptide at one or more amino acid residues from amino acid 1 through amino acid 4, or from amino acid 5 through about 10. In other embodiments, the PEGylated IFN-a is PEGylated at one or more amino acid residues from about 10 to about 28. In other embodiments, the PEGylated IFN-a is PEGylated at or near the carboxyl terminus (C-terminus) of the IFN-a polypeptide, such as at one or more residues from amino acids 156-166, or from amino acids 150 to 155. In other embodiments, the PEGylated IFN-a is PEGylated at one or more amino acid residues at one or more residues from amino acids 100-114. Selection of the attachment site of polyethylene glycol on the IFN-a is determined by the role of each of the sites within the receptor-binding and/or active site domains of the protein, as would be known to the skilled artisan. In general, amino acids at which PEGylation is to be avoided include amino acid residues from amino acid 30 or amino acid 40; and amino acid residues from amino acid 113 to amino acid 149. In some embodiments, PEG is attached to IFN-a via a linking group. The linking group is any biocompatible linking group, where “biocompatible” indicates that the compound or group is non-toxic and may be utilized in vitro or in vivo without causing injury, sickness, disease, or death. PEG can be bonded to the linking group, for example, via an ether bond, an ester bond, a thiol bond or an amide bond. Suitable biocompatible linking groups include, but are not limited to, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine. Methods for making succinimidyl propionate (SPA) and succinimidyl butanoate (SBA) ester-activated PEGs are described in U.S. Pat. No. 5,672,662 (Harris, et al.) and WO 97/03106. Methods for attaching a PEG to an IFN-a polypeptide are known in the art, and any known method can be used. See, for example, Park et al, Anticancer Res, 1: 373-376 (1981); Zaplipsky and Lee, Polyethylene Glycol Chemistry: Biotechnical and Biomedical Applications, J. M. Harris, ed., Plenum Press, NY, Chapter 21 (1992); and U.S. Pat. No. 5,985,265. Pegylated IFN-a is also disclosed in U.S. Pat. Nos. 5,382,657; 5,981,709; 5,985,265; and 5,951,974. Pegylated IFN-a encompasses conjugates of PEG and any of the above-described IFN-a molecules, including, but not limited to, PEG conjugated to interferon alpha-2a (Roferon, Hoffman LaRoche, Nutley, N.J.), where PEGylated Roferon is known as PEGASYSX (Hoffman LaRoche); interferon alpha 2b (Intron, Schering-Plough, Madison, N.J.), where PEGylated Intron is known as PEG-INTRONO (Schering-Plough); interferon alpha-2c (Berofor Alpha, Boehringer Ingelheim, Ingelheim, Germany))

Interferon-beta (IFNβ or IFN-β): A member of the type-1 IFN family signaling through the same receptor (interferon type I receptors) and believed to have similar biologic functions as IFN-a. Interferon-beta is sold under the names of Avonex (Biogen Idec), Rebif (Merck Serono) or CinnoVex (CinnaGen is biosimilar).

Interferon-gamma (IFNγ or IFN-γ): Also referred to as type II IFN is known to signal by a separate receptor, but activates a partially-overlapping signaling pathways as type I interferons and shares their ability to promote the production of the effector cell-attracting chemokines.

### III. TLR3AGONISTS

A TLR3 agonist can be selected from any suitable agent that activates TLR3 and/or the subsequent cascade of biochemical events associated with TLR3 activation in vivo. Assays for detecting TLR3 agonist activity are known in the art and include, for example, the detection of luciferase (luc) production from an NF-κB reporter plasmid, or the induction of endogenous IL-8 (K. Kariko et al., J. Immunol. 2004,172: 6545-49, the disclosures of which is incorporated herein by reference). Assays for detecting TLR3 agonism of test compounds are also described, for example, in PCT publication Nos. WO 03/31573, WO 04/053057, WO 04/053452, and WO 04/094671, the disclosures of each of which are incorporated herein by reference.

Regardless of the particular assay employed, a compound can be identified as an agonist of TLR3 if performing the assay with that compound results in at least a threshold increase of some biological activity known to be mediated by TLR3. Conversely, a compound may be identified as not acting as an agonist of TLR3 if, when used to perform an assay designed to detect biological activity mediated by TLR3, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR3 agonism of a compound in a particular assay. The precise threshold increase of TLR3-mediated biological activity for determining whether a particular compound is or is not an agonist of TLR3 in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for multiple TLRs. Those of ordinary skill in the art can readily determine the appropriate threshold with due consideration of such factors. However, regardless of the particular assay employed, a compound can generally be identified as an agonist of TLR3 if performing the assay with a compound results in at least a threshold increase of some biological activity mediated by TLR3.

Assays employing HEK293 cells transfected with an expressible TLR3 structural gene may use a threshold of, for example, at least a three-fold increase in a TLR3-mediated biological activity (such as NF-κB activation) when the compound is provided at a concentration of, for example, from about 1 μM to about 10 μM for identifying a compound as an agonist of the TLR3 transfected into the cell. However, different thresholds and/or different concentration ranges may be suitable in certain circumstances.

A TLR3 agonist can be an agonistic antibody, an agonistic fragment of such antibodies, a chimeric version of such antibodies or fragment, or another active antibody derivative. TLR3 agonist antibodies useful in this invention may be produced by any of a variety of techniques known in the art. Typically, they are produced by immunization of a non-human animal, such as a mouse, with an immunogen comprising a TLR3 protein or TLR3 peptide. The immunogen may comprise intact TLR3-expressing tumor cells, cell membranes from TLR-3-expressing cells, the full length sequence of the TLR3 protein (produced recombinantly or isolated from a natural source), or a fragment or derivative thereof, typically an immunogenic fragment, for example a portion of the polypeptide comprising an epitope exposed on the surface of cells expressing the TLR3.

In some embodiments, the immunogen comprises a wild-type human TLR3 polypeptide or a immunogenic fragment thereof in a lipid membrane, typically derived from a membrane fraction of a TLR-3 expressing cell. In a specific embodiment, the immunogen comprises whole TLR3 expressing tumor cells, intact or optionally chemically or physically lysed.

The preparation of monoclonal or polyclonal antibodies is well known in the art, and any of a large number of available techniques can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). The step of immunizing a non-human mammal with an immunogen may be carried out in any manner well known in the art for (see, for example, E. Harlow and D. Lane, Antibodies: A Laboratory Manual. Cold Spring Harbor Laboratory Press, Cold Spring Harbor, N.Y. (1988)). Generally, the immunogen is suspended or dissolved in a buffer, optionally with an adjuvant, such as complete Freund's adjuvant. Methods for determining the amount of immunogen, types of buffers and amounts of adjuvant are well known to those of skill in the art and are not limiting in any way on the present invention. Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with adjuvant such as incomplete Freund's adjuvant. The recall injections are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be utilized as long as they result in the production of B cells expressing an antibody directed to the antigen used in immunization. In another embodiment, lymphocytes from an unimmunized non-human mammal are isolated, grown in vitro, and then exposed to the immunogen in cell culture. The lymphocytes are then harvested and the fusion step described below is carried out. For monoclonal antibodies, which are preferred for producing agonistic antibodies for use in the present methods, the next step is the isolation of cells, for example lymphocytes, splenocytes, or B cells, from the immunized non-human mammal and the subsequent fusion of those splenocytes, or B cells, or lymphocytes, with an immortalized cell in order to form an antibody-producing hybridoma. The isolation of splenocytes, such as from a non-human mammal is well-known in the art and generally involves removing the spleen from an anesthetized non-human mammal, cutting it into small pieces and squeezing the splenocytes from the splenic capsule and through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer. Once isolated and present in single cell suspension, the antibody-producing cells are fused to an immortal cell line. This is typically a mouse myeloma cell line, although many other immortal cell lines useful for creating hybridomas are known in the art. Preferred murine myeloma lines include, but are not limited to, those derived from MOPC-21 and MPC-11 mouse tumors available from the Salk Institute Cell Distribution Center, San Diego, Calif. U.S.A., X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

The hybridomas can be grown on a feeder layer of macrophages. The macrophages are preferably from littermates of the non-human mammal used to isolate splenocytes and are typically primed with incomplete Freund's adjuvant or the like several days before plating the hybridomas. Fusion methods are described, e.g., in (Goding, “Monoclonal Antibodies: Principles and Practice,” pp. 59-103 (Academic Press, 1986)), the disclosure of which is herein incorporated by reference. The cells are allowed to grow in the selection media for sufficient time for colony formation and antibody production. This is usually between 7 and 14 days. The supernatants from the hybridoma colonies are then assayed for the production of antibodies that specifically activate the TLR3 protein. The wells positive for the desired antibody production are examined to determine if one or more distinct colonies are present. If more than one colony is present, the cells may be re-cloned and grown to ensure that only a single cell has given rise to the colony producing the desired antibody. Positive wells with a single apparent colony are typically recloned and re-assayed to ensure that only one monoclonal antibody is being detected and produced.

Hybridomas that are confirmed to be producing a TLR3 agonistic monoclonal antibody are then grown up in larger amounts in an appropriate medium, such as DMEM or RPMI-1640. Alternatively, the hybridoma cells can be grown in vivo as ascites tumors in an animal. After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Amersham Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed. In certain embodiments, the DNA encoding an antibody that agonizes TLR3 is isolated from the hybridoma, and placed in an appropriate expression vector for transfection into an appropriate host. The host is then used for the recombinant production of the antibody, variants thereof, active fragments thereof, or humanized or chimeric antibodies comprising the antigen recognition portion of the antibody. DNA encoding the monoclonal antibodies of the invention can be readily isolated and sequenced using conventional procedures (e.g., by using oligonucleotide probes that are capable of binding specifically to genes encoding the heavy and light chains of murine antibodies). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant expression in bacteria of DNA encoding the antibody is well known in the art (see, for example, Skerra et al. (1993) Curr. Op. Immunol. 5:256; and Pluckthun (1992) Immunol. Revs. 130:151.

Antibodies may also be produced by selection of combinatorial libraries of immunoglobulins, as disclosed for instance in Ward et al. (1989) Nature 341:544. The TLR3 agonist antibodies can be full length antibodies or antibody fragments or derivatives. Examples of antibody fragments include Fab, Fab′, Fab′-SH, F(ab′)2, and Fv fragments; diabodies; single-chain Fv (scFv) molecules; single chain polypeptides containing only one light chain variable domain, or a fragment thereof that contains the three CDRs of the light chain variable domain, without an associated heavy chain moiety; single chain polypeptides containing only one heavy chain variable region, or a fragment thereof containing the three CDRs of the heavy chain variable region, without an associated light chain moiety; and multispecific antibodies formed from antibody fragments. Such fragments and derivatives and methods of preparing them are well known in the art. For example, pepsin can be used to digest an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments may be synthesized de novo either chemically or by using recombinant DNA methodology.

A small molecule TLR3 agonist can be an organic molecule of less than about 1500 Daltons. The design and selection (e.g., from a combinatorial library) or synthesis of a small molecule TLR3 agonist may be achieved through the use of the known crystal structure of TLR3 (Choe et al., Science. 309, pp. 581-85 (2005), the disclosure of which is herein incorporated by reference). This design or selection may begin with selection of the various moieties which fill the putative binding pocket(s) in which known double-stranded RNA agonists bind. There are a number of ways to select moieties to fill individual binding pockets. These include visual inspection of a physical model or computer model of the active site elucidated from the crystal structure and manual docking of models of selected moieties into various binding pockets. Modeling software that is well known and available in the art may be used. These include QUANTA [Molecular Simulations, Inc., Burlington, Mass., 1992], and SYBYL [Molecular Modeling Software, Tripos Associates, Inc., St. Louis, Mo., 1992]. This modeling step maybe followed by energy minimization with standard molecular mechanics force fields such as CHARMM and AMBER. (Weiner et al, J. Am. Chem. Soc, 1984, 106, 765; Brooks et al., J. Comp. Chem. 1983, 4, 187). In addition, there are a number of more specialized computer programs to assist in the process of optimally placing either complete molecules or molecular fragments into the TLR3 agonist binding site. These include: GRID (Goodford, P. J. A Computational Procedure for Determining Energetically Favorable Binding Sites on Biologically Important Macromolecules. J. Med. Chem. 1985, 28, 849-857). GRID is available from Oxford University, Oxford, UK; MCSS (Mariner, A.; Karplus, M. Functionality Maps of Binding Sites: A Multiple Copy Simultaneous Search Method. Proteins: Structure, Function and Genetics 1991, 11, 29-34). MCSS is available from Molecular Simulations, Burlington, Mass.; and DOCK (Kuntz, I. D.; Blaney, J. M.; Oatley, S. J.; Langridge, R.; Ferrin, T. E. A Geometric Approach to Macromolecule-Ligand Interactions. J. Mol. Biol. 1982, 161, 269-288). DOCK is available from the University of California, San Francisco, Calif.

Once suitable binding orientations have been selected, complete molecules can be chosen for biological evaluation. In the case of molecular fragments, they can be assembled into a single agonist. This assembly may be accomplished by connecting the various moieties to a central scaffold. The assembly process may, for example, be done by visual inspection followed by manual model building, again using software such as Quanta or Sybyl. A number of other programs may also be used to help select ways to connect the various moieties (Bartlett et al., CAVEAT: A Program to Facilitate the Structure-Derived Design of Biologically Active Molecules. In “Molecular Recognition in Chemical and Biological Problems,” Special Pub., Royal Chem. Soc. 1989, 78, 182-196). In addition to the above computer assisted modeling of agonist compounds, a TLR3 agonist may be constructed “de novo” using either an empty agonist binding site of TLR3 or optionally including some portions of a known agonist. Such methods are well known in the art.

A number of techniques commonly used for modeling drugs may be employed (for a review, see: Cohen et al., “Molecular Modeling Software and Methods for Medicinal Chemistry,” J. Med. Chem., 1990, 33, 883). There are likewise a number of examples in the chemical literature of techniques that can be applied to specific drug design projects. For a review, see: Navia, M. A. and Murcko, M. A., Current Opinions in Structural Biology. 1992, 2, 202. Some examples of these specific applications include: Baldwin, J. J. et al., J. Med. Chem. 1989, 32, 2510; Appelt, K. et al., J. Med. Chem. 1991, 34, 1925; and Ealick, S. E. et al. Proc. Nat. Acad. Sci. USA. 1991, 88, 11540. An alternative to designing or modeling small molecule TLR3 agonists is to screen existing small molecule chemical libraries for a TLR3 agonist. Such libraries can be screened by any TLR3 agonist assay known in the art, including those described herein. The compound libraries may be initially screened using a higher throughput assay, such as a competition assay with a known, labeled TLR3 agonist, such as the double-stranded RNA molecules polyLpolyC or polyA:polyU. Compounds that are positive in a competition assay are then further assayed for their ability to activate TLR3 and cause the subsequent cascade of biochemical events.

Nucleic acid-based TLR3 agonists comprise a region of double-stranded ribonucleic acids. The term “double-stranded” means a portion of the agonist where ribonucleotides are hydrogen bonded (base-paired) to complementary ribonucleotides to form a double-stranded structure. Preferably the entire nucleic acid-based TLR3 agonist consists of ribonucleotides and chemically modified ribonucleotides (“dsRNA TLR3 agonist”). More preferably, at least 50% of the dsRNA TLR3 agonist is in a double-stranded conformation under in vivo conditions. Even more preferred is if at least 60%, 70%, 80%, 90%, 95%, or 97% of the dsRNA TLR3 agonist is in a double-stranded conformation under in vivo conditions. The determination of what percentage of the dsRNA TLR3 agonist is in a double-stranded conformation is achieved by dividing the number of nucleotides that are base-paired by the total number of nucleotides in a molecule. Thus, a 21 base-paired molecule containing 2 nucleotide overhangs at both the 3′ and 5′ end would have 42 nucleotides that are base-paired and 4 nucleotides that are not base-paired, making it 42/46 or 91.3% double-stranded. Similarly, a molecule comprised of two 21 nucleotide strands that are complementary to one another at all nucleotides except for two nucleotides within the middle of each strand would have 38 (19+19) nucleotides that were base-paired and 4 (2+2) that were not base-paired. Such a molecule would be 38/42 or 90.5% double-stranded.

A double-stranded region of a dsRNA TLR3 agonist can be formed by a self-complementary region of a single RNA molecule (for example, a stem and loop structure, such as hairpin RNA and shRNA), by two molecules of RNA that hybridize with one another in whole or in part (as in double-stranded RNA), or a mixture of both (e.g., a partially self-complementary molecule of RNA and a second RNA molecule that hybridizes to regions in the former in that remain single-stranded after the formation of the hairpin). The dsRNA TLR3 agonist can also comprise single-stranded regions, such as 3′ and/or 5′ overhangs at either end of the agonist, and/or “mismatched” or “loop-out” structures within the agonist. In the case of shRNA, the dsRNA TLR3 agonist is encoded by a DNA sequence present on an expression vector. The expression vector is the molecule that is administered to the subject. The expression vector typically comprises a promoter that is activated by RNA polymerase II or E1 and terminator sequences, each of which is operably linked to the shRNA coding sequence to ensure its proper transcription. Promoters activated by RNA polymerase π include, but are not limited to, U6, tRNAval, H1, and modified versions of the foregoing. Promoters activated by RNA polymerase III include, but are not limited to, CMV and EF 1α. In one embodiment, the promoter is an inducible promoter or a tumor cell-specific promoter. Within the context used herein, the term “dsRNA TLR3 agonist” designates any therapeutically or prophylactically effective RNA compound that comprises a double-stranded region. Such compounds are typically active per se; they do not encode a polypeptide or do not require translation to be active. A dsRNA TLR3 agonist can be of any length. Preferably, a dsRNA TLR3 agonist has a length of at least about 10 base pairs (bp), 20 bp, 30 bp, 50 bp, 80 bp, 100 bp, 200 bp, 400 bp, 600 bp, 800 bp or 1000 bp. In one aspect the dsRNA TLR3 agonist is a short dsRNA having a chain length of less than 30 bp, 50 bp, 80 bp,100 bp or 200 bp. In another embodiment, the dsRNA TLR3 agonist is a longer dsRNA, but having a chain length of less than 400 bp, 600 bp, 800 bp or 1000 bp. In another embodiment, the dsRNA TLR3 agonist is a long dsRNA having a chain length of greater than 1000 bp. In one aspect, a dsRNA TLR3 agonist is a composition that comprises a heterogeneous mixture of dsRNA molecules, wherein a plurality of molecules have differing lengths. Preferably the dsRNA molecules in such a composition have on average a length of at least about 10 bp, 20 bp, 30 bp, 50 bp, 80 bp, 100 bp, 200 bp, 400 bp, 600 bp, 800 bp or 100 bp. In another embodiment, a dsRNA TLR3 agonist composition comprises a plurality dsRNA molecules where at least 20%, 50%, 80%, 90% or 98% of dsRNA molecules have a length of at least about 10 bp, 20 bp, 30 bp, 50 bp, 80 bp, 100 bp, 200 bp, 400 bp, 600 bp, 800 bp or 1000 bp per strand. In one example, the dsRNA is a short dsRNA having between 10 and 30, more preferably between 20 and 30 bp per strand. In another example, a dsRNA TLR3 agonist composition has a substantially homogenous mixture of dsRNA molecules, where substantially all the molecules on each strand do not differ in chain length by more than 30 bp, 50 bp, 80 bp, 100 bp or 200 bp. Average chain length of dsRNA TLR3 agonists in a composition can be determined easily, for example, by gel permeation chromatography. One or more of the dsRNA molecules within such a compositions is optionally a siRNA molecule targeted against a cancer antigen. The ribonucleotides in the dsRNA TLR3 agonist can be natural or synthetic, and may be chemically modified derivatives or analogs of natural nucleotides. Modifications include are stabilizing modifications, and thus can include at least one modification in the phosphodiester linkage and/or on the sugar, and/or on the base. For example, one or both strands of the dsRNA can independently include one or more phosphorothioate linkages, phosphorodithioate linkages, and/or methylphosphonate linkages; modifications at the 2′-position of the sugar, such as 2′-O-methyl modifications, 2′-O-methoxyethyl modifications, 2′-amino modifications, 2′-deoxy modifications, 2′-halo modifications such as 2′-fluoro; combinations of the above, such as 2′-deoxy-2′-fluoro modifications; acyclic nucleotide analogs, and can also include at least one phosphodiester linkage.

Oligonucleotides used in the dsRNA TLR3 agonist can also include base modifications or substitutions. Modified bases include other synthetic and naturally-occurring bases such as 5-methylcytosine (5-Me-C), 5-hydroxymethyl cytosine, xanthine, hypoxanthine, 2-aminoadenine, 6-methyl and other alkyl derivatives of adenine and inosine, 2-propyl and other alkyl derivatives of adenine and inosine, 2-thiouracil and 2-thiocytosine, 5-halouracil and cytosine, 5-propynl(—C═C—CH₃) uracil and cytosine and other alkynyl derivatives of pyrimidine bases, 6-azo uracil and cytosine, 5-uracil (pseudouracil), 4-thiouracil, 8-halo, 8-amino, 8-thiol, S-thioalkyl, 8-hydroxyl and other 8-substituted adenines and inosines, 5-halo particularly 5-bromo, 5-trifluoromethyl and other 5-substituted uracils and cytosines, 7-methylinosine and 7-methyladenine, 2-F-adenine, 2-amino-adenine, 8-azainosine and 8-azaadenine, 7-deazainosine and 7-deazaadenine and 3-deazainosine and 3-deazaadenine.

Other modifications include a 3′- and/or 5′-terminal cap, a terminal 3′-5′ linkage, and a 5′-terminal phosphate group or modified phosphate group. Examples of terminal cap moieties include, but are not limited to, an inverted deoxy abasic moiety, an inverted deoxynucleotides, or a glyceryl moiety. In addition, one or both strands (in a two-stranded dsRNA TLR3 agonist) can be or include a concatemer consisting of two or more oligonucleotide sequences joined by a linker(s). All of these modifications are well known in the art (see, for example, Kandimalla et al. ((2003) Nucl. Acid. Res. 31(9): 2393-2400). Previous studies of double-stranded RNA (dsRNA) assessing their ability to be effective interferon inducers suggested that dsRNA agents must possess the secondary structure of a double stranded helix. Other dsRNA agents which have also been shown to be suitable as TLR3 agonist include double-stranded polynucleotides which are not complementary or not perfectly complementary; these have been known as, so-called “mismatched” or “loop-out” structures and exist in naturally occurring RNAs such as transfer tRNAs, ribosomal RNAs and the viral RNA secondary structures. One commonly cited dsRNA compound, Ampligen, comprises a structure where a few parts of cytidine in the poly Lpoly C structure are replaced with uridine (i.e. mismatched RNA); this compound has been reported to have physiological activity similar to that of the parent poly Iipoly C. However it will be appreciated that TLR3 agonists of any type and configuration can be used in accordance with the disclosed methods. Generally, the polynucleotides need to be resistant to nucleases in order to remain as macromolecules for a sufficient length of time; polynucleotides are less sensitive to nuclease attack when they are in a helical complex. However, certain analogs such as Ampligen™ appear to retain their TLR3 agonist activity. In a particular embodiment, each strand of these dsRNAs can have a length comprised between about 5 and 50 bases, more preferably between 5 and 40, 35, 30, 25 or 20 bases. Each strand is preferably perfectly complementary to the other. Preferred examples of such dsRNAs are homopoly RNAs, such as dsRNAs in which each strand comprises essentially a repeat of the same base; or comprises a homopolyRNA region.

The base in such homopoly RNA strands may be any naturally occurring base (e.g., polyA, polyU, polyC, polyG) or non-naturally occurring (e.g., chemically synthesized or modified) base (e.g., polyl). Polynucleotides typified by polyinosinic-polycytidylic acid, i.e., poly(I):poly(C) or poly LC and polyadenylic-polyuridylic acid, i.e., poly(A):poly(U) or poly A:U, are well-known compounds in the art and have been known to induce interferon production by immune cells. Thus in some embodiments, the TLR3 agonist for use according to the invention is a double stranded RNA selected from the group consisting of: polyinosinic acid and polycytidylic acid, polyadenylic acid and polyuridylic acid, polyinosinic acid analogue and polycytidylic acid, polyinosinic acid and polycytidylic acid analogue, polyinosinic acid analogue and polycytidylic acid analogue, polyadenylic acid analogue and polyuridylic acid, polyadenylic acid and polyuridylic acid analogue, and polyadenylic acid analogue and polyuridylic acid analogue. The term “analogue” as used herein means any of the nucleotide modifications described above. It will be appreciated that dsRNA TLR3 agonists can comprise any combination of bases and be designed using any suitable method. Preferably, the basic requirement of a region of double-strandedness, stability and resistance to nuclease attack and the preferences for chain length are taken into account. These properties, as well as relative TLR3 agonistic activity of any dsRNA TLR3 agonist can be tested and assessed with reference to the a rAₙ:rUₙ or rIₙ:rCₙ complex for example. Measures can be taken to increase stability and resistance to nucleases, or to increase or optionally decrease interferon-inducing action.

Other examples of dsRNA include nucleic acids described in U.S. Pat. Nos. 5,298,614 and 6,780,429. U.S. Pat. No. 5,298,614 discloses that when chain length of the double stranded nucleic acid derivatives is limited to certain ranges, the resulting substances exhibit desired physiological activity with markedly less toxicity, providing polynucleotides having a length of about 50 to 10,000 as calculated by base pair numbers. Also described are derivative wherein the purine or pyrimidine ring in the nucleic acid polymer is substituted with at least one SH group, or said derivative contains a disulphide bond, or both (preferred ratio of number of sulphur atoms to cytidylic acid present in the poly C are 1:6 to 39). U.S. Pat. No. 6,780,429 describes a particular type of dsRNA compounds that are “chain-shortened” having lengths of about 100 to 1,000 as calculated by base pair numbers, or preferably from 200 to 800, and more preferably from 300 to 600. The latter compounds are reported to contain low numbers of 2′-5′ phosphodiester bonds by a method designed to avoid phosphate groups causing intramolecular rearrangement from 3′ position to 2′ position through a mechanism called pseudo rotation simultaneously that can occur during hydrolysis of polynucleotides, resulting in a portion of 3′-5′ phosphodiester bonds in the chain-shortened polynucleotide molecule being replaced by 2′-5′ phosphodiester bonds.

Other nucleic acid agonists that can be suitable for use as TLR3 agonists are provided in: Field et al: Proc. Nat. Acad. Sci. U.S. 58, 1004, (1967); Field et al: Proc. Nat. Acad. Sci. U.S. 58, 2102, (1967); Field et al: Proc. Nat. Acad. Sci. U.S. 61, 340, (1968); Tytell et al: Proc. Nat. Acad. Sci. U.S. 58, 1719, (1967); Field et al: J. Gen. Physiol. 56, 905 (1970); De Clercq et al: Methods in Enzymology, 78, 291 (1981). A number of synthetic nucleic acid derivatives have been described, including homopolymer-homopolymer complexes (Double Strand Nucleic Acid Polymer such as those in which poly I:C or poly A:U are a parent structure, where these homopolymer-homopolymer complexes contain: (1) base modifications, exemplified by Polyinosinic acid-poly(5-bromocytidylic acid), Polyinosinic acid-poly(2-thiocytidylic acid), Poly(7-deazainosinic acid)-polycytidylic acid, Poly(7-deazainosinic acid)-poly(5-bromocytidylic acid), and Polyinosinic acid-poly(5-thiouridylic acid); (2) Sugar Modifications, exemplified by Poly(2′-azidoinosinic acid)-polycytidylic acid; and (3) Phosphoric Acid Modifications, exemplified by Polyinosinic acid-poly(cytidyl-5′-thiophosphoric acid). Other synthetic nucleic acid derivatives that have been described include interchanged copolymers, exemplified by Poly(adenylic acid-uridylic acid); and homopolymer-copolymer complexes, exemplified by Polyinosinic acid-poly(cytidylic acid-uridylic acid) and Polyinosinic acid-poly(citydylic acid-4-thiouridylic acid). Other synthetic nucleic acid derivatives that have been described include complexes of synthetic nucleic acid with polycation, exemplified by Polyinosinic acid-polycytidylic acid-poly-L-lysinecarboxy-methylcellulose complex (called “Poly ICLC”). Yet another example of synthetic nucleic acid derivative is Polyinosinic acid-poly(1-vinylcytosine).

One example of a TLR3 agonist is AMPLIGEN™ (Hemispherx, Inc., of Rockville, Md., U.S.A.), a dsRNA formed by complexes of polyriboinosinic and polyribocytidylic/uridylic acid, such as rIₙ:r(Cₓ,U or G)ₙ where x has a value from 4 to 29, e.g., rIₙ:r(Cj2 U)n-. Many mismatched dsRNA polymers which behave similarly to AMPLIGEN™ have been studied; mismatched dsRNA based on poly LC have included complexes of a polyinosinate and a polycytidylate containing a proportion of uracil bases or guanidine bases, for example from 1 in 5 to 1 in 30 such bases. The key therapeutic advantage of mismatched dsRNAs over other forms of natural and/or synthetic dsRNAs is a reported reduction in toxicity over compounds such as those described in U.S. Pat. No. 3,666,646. Specific examples of double-stranded RNA according to the present invention further include Polyadenur (Ipsen) and Ampligen (Hemispherx). Polyadenur is a polyA/U RNA molecule, i.e., contains a polyA strand and a polyU strand. Polyadenur has been developed for the potential treatment of hepatitis B virus (HBV) infection. AMPLIGEN™ is of a polyl/polyC compound (or a variant thereof comprising a polyl/polyC12U RNA molecule). AMPLIGEN™ is disclosed for instance in EP 281 380 or EP 113 162. AMPLIGEN™ has been proposed for the treatment of cancer, viral infections and immune disorders. It was developed primarily for the potential treatment of myalgic encephalomyelitis (ME, or chronic fatigue syndrome/chronic fatigue immune dysfunction syndrome, CFS/CFIDS).

A particular example of a dsRNA for use is a dsRNA comprising a polyA/polyU region, wherein each strand of said dsRNA contains less than 25 bases. Another particular example of a dsRNA for use is a dsRNA comprising a polyl/polyC(U) region, wherein each strand of said dsRNA contains less than 25 bases. Further dsRNAs have been disclosed in the literature or may be developed, which can be used within the present methods. More generally, any synthetic double-stranded homopolyRNA can be used, as well as any other dsRNA as herein described. In one embodiment, the dsRNA is a fully double stranded (e.g., blunt-ended; no overhangs) polyA:polyU molecule consisting of between 19 and 30 base pairs (e.g., 19, 20, 21, 22, 23, 24, 25, 26, 27, 28, 29 or 30 base pairs) and comprising between 1 and 30 stabilizing modifications and/or a 3′ and/or a 5′ cap. Stabilizing modification and caps are described above and are well-known in the art. The stabilizing modifications and/or the presence of a cap make the dsRNA more resistant to serum degradation. Stabilizing modifications include phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications are known to dramatically increase the serum stability of dsRNA compounds. One example of a stabilized dsRNA is STEALTH™ RNAi (commercially available from Invitrogen, Carlsbad, Calif. USA). Another example of stabilized dsRNA is poly-ICLC (Hiltonol, produced by Oncovir).

In another embodiment the dsRNA TLR3 agonist is a siRNA molecule or a shRNA molecule that is designed to specifically hybridize with the mRNA coding a tumor cell antigen or another protein involved in tumor proliferation. In this embodiment, the dsRNA molecule plays a dual role in the treatment of cancer. It is both an agonist of TLR3 and a suppressor of a specific tumor antigen expression. It has been demonstrated that siRNA molecules and shRNA molecules targeted against cellular proteins exhibit both sequence-dependent gene suppression and sequence-independent effects mediated through TLR3 (K. Kariko et al., J. Immunol. 2004, 172: 6545-6549). Thus, it is expected that tumor antigen- or tumor-proliferation specific siRNA and shRNA molecules will also be agonists of TLR3 in cancer cells.

Ligands of alternative Toll-like receptors 3 (TLRs 1-9) also known to activate NF-kB pathway: There have been a total of 13 TLRs identified in mammals, including nine (TLR1-9) that have been expensively studied and are known to activate the NF-κB pathway.

Activated TLRs recruit adapter molecules within the cell cytoplasm to initiate signal transduction. At least four adapter molecules, MyD88, TIRAP (Mal), TRIF, and TRAM are known to be involved in signaling.

TLR signaling is divided into two distinct signaling pathways, the MyD88-dependent and TRIF-dependent pathway. The MyD88-dependent response occurs on dimerization of the TLR receptor, and is utilized by every TLR except TLR3. The primary effect of MyD88 activation is the activation of NF-κB. MyD88 (a member of TIR family) recruits IRAM kinases IRAK 1, IRAK 2, and IRAK 4. IRAK kinases phosphorylate and activate the signaling protein TRAF6, which in turn polyubiquinates the protein TAK1, as well as itself in order to facilitate binding to IKKβ. On binding, TAK1 phosphorylates IKKβ, which then phosphorylates IκB causing its degradation and allowing NF-κB to enter the cell nucleus and activate transcription.

Both TRL3 and TRL4 utilize the TRIF-dependent pathway, which is triggered, respectively, by dsRNA and LPS. For TRL3, dsRNA leads to activation of the receptor, recruiting the adaptor TRIF. TRIF activates the kinases TBK1 and RIP1. The TRIF/TBK1 signaling complex phosphorylates IRF3, promoting its entry into the nucleus and production of type I IFNs. The activation of RIP 1 causes the polyubiquination and activation of TAK1 (joint pathway with MyD88 signaling and NF-κB transcription, similar to the MyD88-dependent pathway of other TLR signaling.

### IV. PROSTAGLANDIN SYNTHESIS INHIBITORS

Prostaglandins, particularly prostaglandin E2 (PGE2), are involved in many diverse physiological and pathophysiological functions. These eicosanoids are produced by the action of prostaglandin endoperoxide synthase on arachidonic acid. Prostaglandin endoperoxide synthase activity originates from two distinct and independently regulated isozymes, termed as prostaglandin endoperoxide synthase-1 and prostaglandin endoperoxide synthase-2 and are encoded by two different genes.

Prostaglandin endoperoxide synthase-1 is expressed constitutively and is thought to play a physiological role, particularly in platelet aggregation, cytoprotection in the stomach, and regulation of normal kidney function. Prostaglandin endoperoxide synthase-2 (PGE2) is the inducible isozyme and expression of prostaglandin endoperoxide synthase-2 is induced by a variety of agents which include endotoxin, cytokines, and mitogens. Importantly, prostaglandin endoperoxide synthase-2 is induced in vivo in significant levels upon pro-inflammatory stimuli.

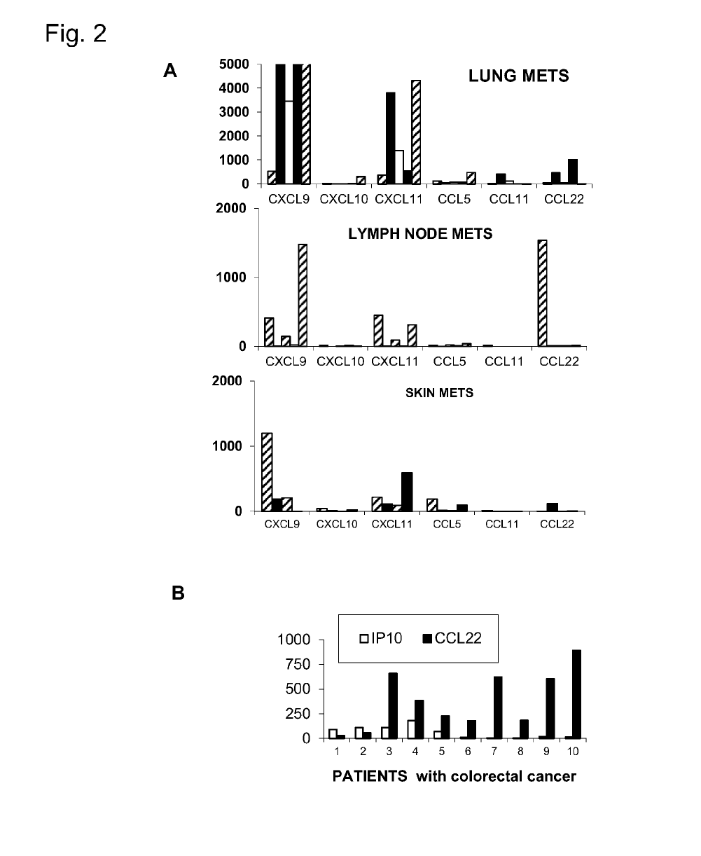

Two general structural classes of prostaglandin endoperoxide synthase-2 selective inhibitors are commonly reported in the literature. In addition to selective prostaglandin endoperoxide synthase-2 inhibition in vitro, many of these compounds possess potent anti-inflammatory activity in the rat adjuvant-induced arthritis model along with exceptional safety profiles in comparison with the existing anti-inflammatory agents. The structural classes include the tricyclic non-acidic arylmethyl sulfones (exemplified by DuP 697 and SC 8092) and the acidic sulfonamides (exemplified by Flosulide and NS-398) (FIG. 2). The arylmethyl sulfonyl moiety in the tricyclic non-acidic compounds such as SC 8092 may play a key role in the selective prostaglandin endoperoxide synthase-2 inhibition by these compounds as reduction of the sulfone group in SC 8092 to the corresponding sulfide functionality generates SC 8076, a prostaglandin endoperoxide synthase-1 selective inhibitor.

PGE2 inhibitors include Cox-2 inhibitors. Suitable COX-2 inhibitors for use in the invention may include the following compounds or derivatives thereof or a pharmaceutically acceptable salt thereof, or any hydrate thereof: rofecoxib, etoricoxib, celecoxib, valdecoxib, parecoxib. An alternative class of Cox-2 inhibitors compounds for use in the invention is the methane sulfonanilide class of inhibitors, of which NS-398, flosulide, nimesulide are example members. A further class of COX-2 inhibitors is the tricyclic inhibitor class, which can be further divided into the sub-classes of tricyclic inhibitors with a central carbocyclic ring (examples include SC-57666, 1 and 2; those with a central monocyclic heterocyclic ring (examples include DuP 697, SC-58125, SC-58635, SC 236 and 3,4 and 5); and those with a central bicyclic heterocyclic ring (examples include 6, 7, 8, 9 and 10). Compounds 3, 4, and 5 are described in U.S. Pat. No. 5,474,995. A yet further class of COX-2 inhibitors can be referred to as those which are structurally modified NSAIDS. In addition to the structural classes, sub-classes, specific COX-2 inhibitors compound examples, examples of compounds which selectively inhibit cyclooxygenase-2 have also been described in the following patent publications, all of which are herein incorporated by reference: U.S. Pat. Nos. 5,344,991; 5,380,738; 5,393,790; 5,409,944; 5,434,178; 5,436,265; 5,466,823; 5,474,995; 5,510,368; 5,536,752; 5,550,142; 5,552,422; 5,604,253; 5,604,260; 5,639,780; and International Patent Specification Nos. 94/13635, 94/15932, 94/20480, 94/26731, 94/27980, 95/00501, 95/15316, 96/03387, 96/03388, 96/06840; and International Publication No.'s WO 94/20480, WO 96/21667, WO 96/31509, WO 96/36623, WO 97/14691, WO 97/16435. Some of the compounds above can also be identified by the following chemical names: 3: 3-phenyl(4-(methylsulfonyl)phenyl)-2-(5H)-furanone; 4: 3-(3,4-difluorophenyl)-4-(4-(methylsulfonyl)phenyl)-2-(5H)-faranone; 5: 5,5-dimethyl-4-(4-(methylsulfonyl)phenyl)-3-(3-fluorophenyl)-H-furan-2-1-one; 12: 5,5-dimethyl-4-(4(methylsulfonyl)phenyl)-3-(2-propoxy)-5H-furan-2-one; 13: 5-chloro-3-(4-(methylsulfonyl)phenyl)-2-(2-methyl-5-pyridinyl)pyridine; 14: 2-(3,5-difluorophenyl)-3-(4-(methylsulfonyl)phenyl)-2-cyclopenten-1-one; 15: 5(S)-5-ethyl-5-methyl-4-(4-methylsulfonyl)phenyl)-3-(2-propoxy)-5H-furan-2-one; 16: 5-ethyl-5-methyl-4-(4-(methylsulfonyl)phenyl)-3-(3,4-difluorophenyl)-5H-furan-2-one; 17: 342-thiazolyl)methoxy)-4-(4-methylsulfonyl)phenyl)-5,5-dymethyl-5H-furan-2-one; 18: 3-propyloxy-4-(4-methylsulfonyl)phenyl)-5,5-dimethyl-5H-furan-2-one; 19: 3-(1-cyclopropylethoxy)-5,5-dimethyl-4-(4-methylsulfonyl)phenyl)-5H-furan-1-2-one; 20: sodium 2-(4-chlorophenyl)-3-(4-methylsulfonyl)phenyl)-4-oxo-2-pentenoate; 21: 3-(cyclopropylmethoxy)-5,5-dimethyl-4(4-methylsulfonyl)phenyl)-5H-furan-2-1-one; 22: 3-(cyclopropylmethoxy)-5,5-dimethyl-4-(4-methylsulfonyl)phenyl)-2,5-dihydrofuran-2-ol; 23: 33-isopropoxy-5,5-dimethyl-4-(4-methylsulfonyl)phenyl)-2,5-dihydrofuran-2-1-ol; 24: 5,5-dimethyl-3-(3-fluorophenyl)-2-hydroxy-4-(4-methylsulfonyl)phenyl)-2,5-dihydrofuran; 25: 5-Chloro-3-(4-methylsulfonyl)phenyl)-2-(3-pyridinyl)pyridine. See also U.S. Pat. No. 7,345,088, incorporated herein by reference.

Examples of the most commonly used selective COX2 inhibitors include celecoxib, alecoxib, valdecoxib, and rofecoxib.

Examples of the most commonly used non-selective COX 1 and COX2 inhibitors include: acetylsalicylic acid (aspirin) and other salicylates, acetaminophen (Tylenol), ibuprofen (Advil, Motrin, Nuprin, Rufen), naproxen (Naprosyn, Aleve), nabumetone (Relafen), or diclofenac (Cataflam).

### V. PROSTAGLANDIN RESPONSIVENESS INHIBITORS

The key suppressive and tumor-promoting effects of prostaglandins are mediated by the activation of adenylate cyclase, the resulting elevation of the intracellular cyclic (c)AMP, PKA and the downstream activation of the PKA/CREB pathway.

Another level of interference with the PG responsiveness includes the interference with their binging to PG receptors. In case of PGE2, the two key cAMP-activating receptors are EP2 and EP4, for which a number of specific inhibitors exist.

The increase of cAMP levels induced by prostaglandins or other factors can be prevented by phosphodiesterases (PDEs; currently known 6 types, PDE1-PDE5 and PDE10, which reduce the levels of intracellular cAMP). PDEs can be controlled by phosphodiesterase inhibitors, which include such substances as xanthines (caffeine, aminophylline, IBMX, pentoxyphylline, theobromine, theophylline, or paraxanthine), which all increase the levels of intracellular cAMP, and the more selective synthetic and natural factors, including vinpocetine, cilostazol, inaminone, cilostazol, mesembrine, rolipram, ibudilast, drotaverine, piclamilast, sildafenil, tadalafil, verdenafil, or papaverine.

Furthermore, interference with PGE2 signaling (or with the signaling of other cAMP-elevating factors, such as histamine, of beta-adrenergic agonists) can be achieved by the inhibition of downstream signals of cAMP, such as PKA or CREB.

### VI. COMPOSITIONS AND THERAPEUTIC METHODS

Methods are disclosed herein for preventing or treating cancer and other diseases, including inflammation, autoimmunity, transplant rejection and graft versus host disease (GvH). Examples of the types of cancers that the disclosed methods can apply to include the following, without limitation: colorectal cancer, lung cancer, laryngeal cancer, melanoma, non-melanoma skin cancers, glioma, ovarian cancer, breast cancer, endometrial cancer, cervical cancer, gastric cancer, esophageal cancer, pancreatic cancer, biliary cancer, renal cancer, bladder cancer, vulvar cancer, neuroendocrine cancer, prostate cancer, head and neck cancer, soft-tissue sarcomas, bone cancer, mesothelioma, cancer of endothelial origin, hematologic malignancy including but not limited to multiple myeloma, lymphomas, leukemias, or a pre-malignant lesion known to be associated with an increased risk of developing cancer. The methods include administering a therapeutically effective amount of agents that increase the production of IP10/CXCL10, MIG/CXCL9, RANTES/CCL5 and other pro-inflammatory chemokines. Such methods include the administration of therapeutically effective amounts of a Toll-like receptor agonist or alternative activators of the NF-κB pathway, combined with the administration of a therapeutically effective amount of an inhibitor prostaglandin synthesis or cAMP-dependent prostaglandin signaling, with the administration of a therapeutically effective amount of an interferon, or both, resulting in binary adjuvants or tertiary adjuvants.

An amount of a therapeutic agent is considered effective if it together with one or more additional therapeutic agents, induces the desired response, such as decreasing the risk of developing cancer or decreasing the signs and symptoms of cancer. In one example, it is an amount of an agent needed to prevent or delay the development of a tumor, in a subject. In another example, it is an amount of the agent needed to prevent or delay the metastasis of a tumor, cause regression of an existing tumor, or treat one or more signs or symptoms associated with a tumor in a subject, such as a subject having melanoma or colorectal cancer. Ideally, a therapeutically effective amount provides a therapeutic effect without causing a substantial cytotoxic effect in the subject. The preparations disclosed herein are administered in therapeutically effective amounts.

In one example, a desired response is to prevent the development of a tumor. In another example, a desired response is to delay the development, progression, or metastasis of a tumor, for example, by at least about 3 months, at least about six months, at least about one year, at least about two years, at least about five years, or at least about ten years. In a further example, a desired response is to decrease the occurrence of cancer, such as colorectal cancer or melanoma. In another example, a desired response is to decrease the signs and symptoms of cancer, such as the size, volume, or number of tumors or metastases. For example, the composition can in some examples decrease the size, volume, or number of tumors (such as colorectal tumors) by a desired amount, for example by at least 5%, at least 10%, at least 15%, at least 20%, at least 25%, at least 30%, at least 50%, at least 75%, or even at least 90%, as compared to a response in the absence of the therapeutic composition.

Compositions are provided that include one or more of the agents disclosed herein that are disclosed herein in a carrier. The compositions can be prepared in unit dosage forms for administration to a subject. The amount and timing of administration are at the discretion of the treating physician to achieve the desired purposes. The agent can be formulated for systemic or local (such as intra-tumor) administration. In one example, the agents are formulated for parenteral administration, such as intravenous administration.

The compositions for administration can include a solution of the agents of use dissolved in a pharmaceutically acceptable carrier, such as an aqueous carrier, or bio-compatible formulations of liposomes or other bio-compatible vesicles, or other slow release matrices and vehicles. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical pharmaceutical composition for intravenous administration includes about 0.1 to 10 mg of antibody per subject per day. Dosages from 0.1 up to about 100 mg per subject per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed., Mack Publishing Company, Easton, Pa. (1995).

Agents such as proteins may be provided in lyophilized form and rehydrated with sterile water before administration, although they are also provided in sterile solutions of known concentration. The protein solution is then added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of RITUXAN® in 1997. Agents can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

The agents can be administered to slow or inhibit the growth of cells, such as cancer cells. In these applications, a therapeutically effective amount of an antibody is administered to a subject in an amount sufficient to inhibit growth, replication or metastasis of cancer cells, or to inhibit a sign or a symptom of the cancer, such as melanoma or colorectal cancer. In some embodiments, the agents are administered to a subject to inhibit or prevent the development of metastasis, or to decrease the number of micrometastases, such as micrometastases to the regional lymph nodes (Goto et al., Clin. Cancer Res. 14(11):3401-3407, 2008).

A therapeutically effective amount of the agents of use will depend upon the severity of the disease and the general state of the patient's health. A therapeutically effective amount of the agent when administered to a subject that has colorectal cancer or melanoma is that which provides either subjective relief of a symptom(s) or an objectively identifiable improvement as noted by the clinician or other qualified observer. These compositions can be administered in conjunction with another chemotherapeutic agent, either simultaneously or sequentially.

Many chemotherapeutic agents are presently known in the art. These can be administered in conjunction with the disclosed methods. In one embodiment, the chemotherapeutic agents is selected from the group consisting of mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones, e.g. anti-androgens, and anti-angiogenesis agents.

Single or multiple administrations of the compositions are administered depending on the dosage and frequency as required and tolerated by the patient. The dosage can be administered once but may be applied periodically until either a therapeutic result is achieved or until side effects warrant discontinuation of therapy. In one example, a dose of the agents is infused for thirty minutes every other day. In this example, about one to about ten doses can be administered, such as three or six doses can be administered every other day. In a further example, a continuous infusion is administered for about five to about ten days. The subject can be treated at regular intervals, such as monthly, until a desired therapeutic result is achieved. Generally, the dose is sufficient to treat or ameliorate symptoms or signs of disease without producing unacceptable toxicity to the patient.

The optimal activity of drugs frequently requires their prolonged administration, and in case of the combination administration of different drugs, it may require their administration in a specific sequence. Both of these requirements can be fulfilled by the application of controlled delivery systems, releasing one, three or more of the components of the treatment with similar or different kinetics, starting at the same time point or sequentially.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992) both of which are incorporated herein by reference.

Polymers can be used for ion-controlled release of the compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342 and U.S. Pat. No. 5,534,496).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### VII. NON-LIMITING EXAMPLES

This disclosed invention is illustrated by the following non-limiting examples.

### Example 1

### Effector-Type Immune Cells Express High Levels of CCR5 and CXCR3

We have observed that DC maturation in the combined presence of IFNα and IFNγ results in the development of stable type-1-polarized DCs (DC1s) with strongly elevated, rather than “exhausted” ability to produce IL-12p70 upon subsequent stimulation. The induction of DC1s in our original protocols depended on the presence of bovine serum.

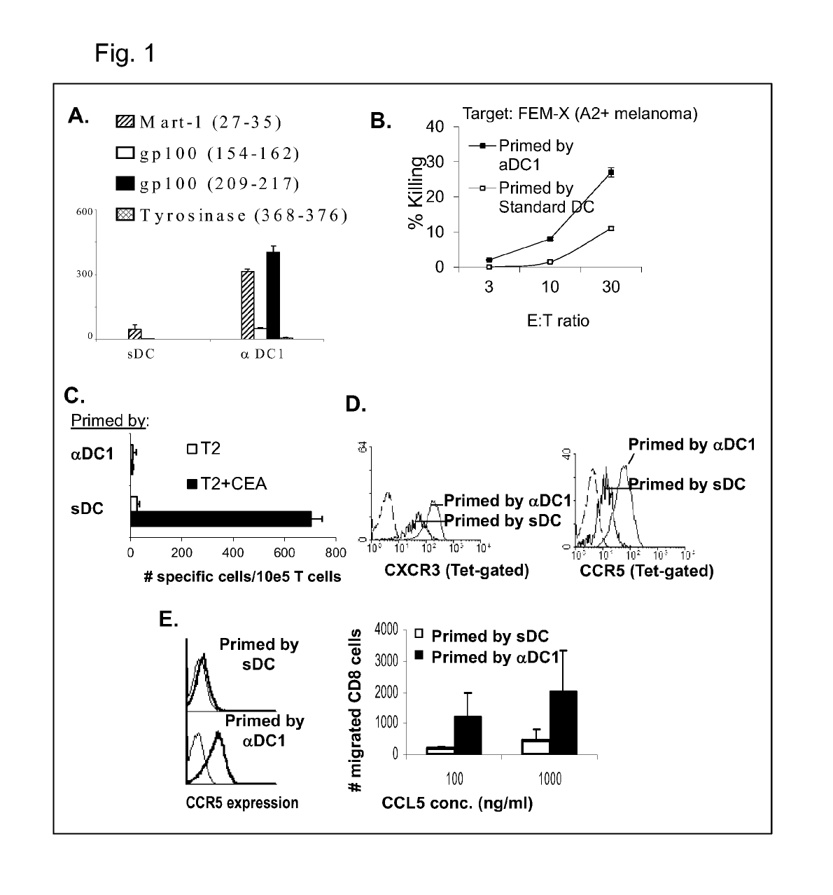

The addition of IFNα and poly-I:C (a synthetic analogue of dsRNA with IFNα-inducing activity), to our basic protocol of DC1 generation (IL-1β/TNFα/IFNγ) allows for the development of DC1 (i.e. αDC1) in clinically-acceptable serum free media. Using the blood of melanoma and colorectal cancer patients, we observed that DC1s induce superior expansion of melanoma- or CRC-specific IFNγ-producing CTLs, capable of recognizing defined CRC- or melanoma-specific epitopes and the relevant tumor cells (FIG. 1A-C). Our data demonstrate that αDC1-induced CTLs are functional also with respect to their ability to kill HLA-A2⁺ cancer cells (FIG. 1B), and show strongly-elevated (ca 10-fold) expression of tumor homing-relevant chemokine receptors: CXCR3 and CCR5 (FIG. 1D). Furthermore, we have obtained functional data (in polyclonal models) showing that the differences in CKR expression between αDC1- and sDC-primed CTLs lead to differential CK responsiveness (FIG. 1E).

### Example 2

### The Expression of Effector T Cell (T_{Eff})-Recruiting Chemokines in Colorectal Tumor Samples Correlates with Effector CD8⁺ T Cell Markers, while COX2 Levels Closely Correlate with the Over-Expression of Treg-Attracting CKs and Suppression of Teff-Attracting CKs and Treg Markers

Freshly-obtained untreated tumors, including melanoma and colorectal cancer lesions show highly heterologous expression of the “desirable” chemokines (CCR5 ligands and CXCR3 ligands) and “undesirable” CCL22 (FIG. 2). Importantly, even within the group of tumors of the same histologic type (melanoma or CRC) and similar location, different tumor samples showed significant heterogeneity with regard to the expression of the individual CKs. Although the numbers of the available samples are relatively small (precluding formal statistical comparison between primary- and metastatic lesions), liver-metastatic CRC showed a possible bias towards the Teff-low/Treg-high CK pattern (data not shown).

These data suggest that some tumors may be able to avoid the immune attack by excluding the Teffs (Teff infiltration has been shown to predict long-term relapse-free survival in resected CRC patients) and preferentially attracting Treg cells, by virtue of a particular chemokine production pattern. Furthermore, they implicate that the standardized overexpression of Teff-attracting chemokines in all tumor lesions may improve the outcomes of spontaneously-arising antitumor-responses and may facilitate the immunotherapy of cancer by selective directing Teff cells to tumor lesions. They implicate that the cancer vaccines that are effective in inducing the desirable effector-type immune cells (known to mainly express CXCR3 and CCR5) may be able to induce regression of only a part of the tumors (these that express CXCR3- and CCR5 ligands), but may need a combination with tumor-specific CK modulatory regimens in order to induce the regression of additional tumors that do not attract Teff cells spontaneously, instead over-expressing Treg-attracting CKs.

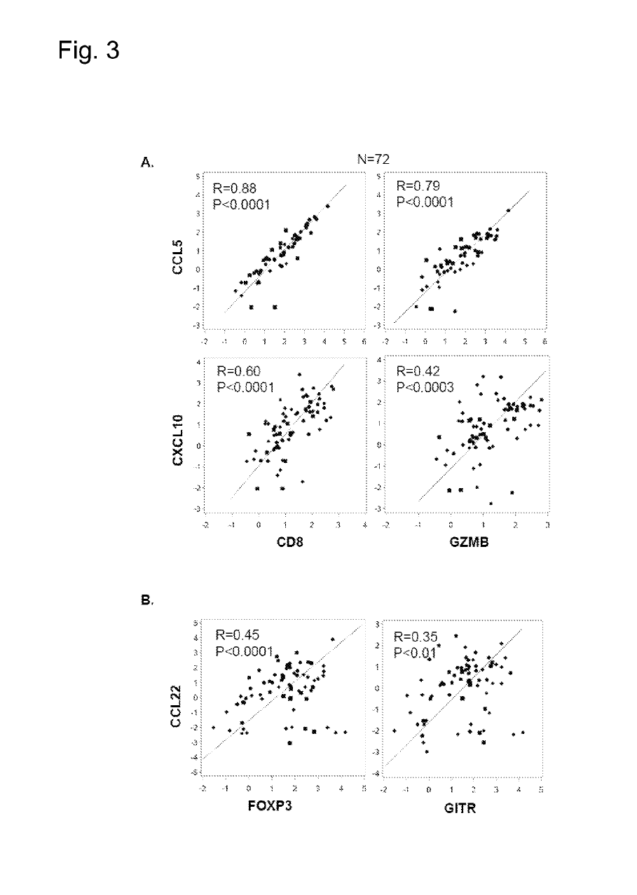

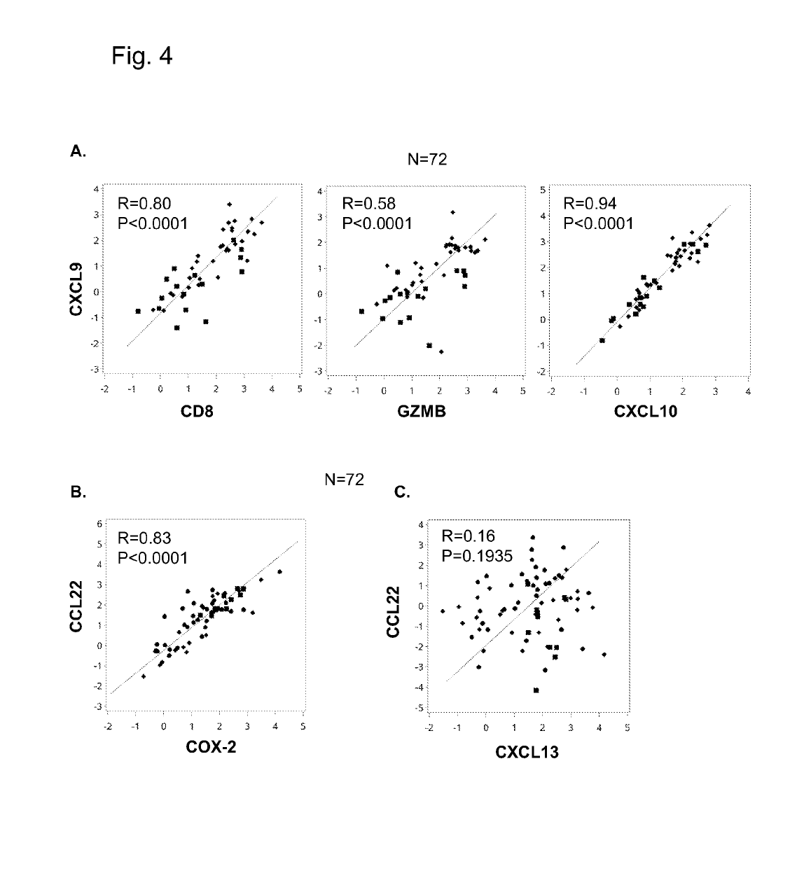

Using resected tumor material from 72 patients with advanced colorectal cancer (metastatic in 68 patients), we observed that local expression of two T_{eff} cell markers (CD8 and Granzyme B; GZMB) is strongly correlated with the expression of two T_{eff}-attracting chemokines, CCL5 and CXCL10 (FIG. 3A). In contrast, the T_{reg} markers FOXP3 and GITR were correlated with CCL22 (FIG. 3B), a known T_{reg} attractant. Additional correlations were observed between CXCL9 (alternative CXCR3 ligand) and T_{eff} markers and between CCL22 and the CCL22-inducing factor (22) COX2 (FIG. 4).

The phenotypic analysis of CD8⁺ tumor-infiltrating lymphocytes (TIL) obtained from such colon cancer patients revealed that the majority of CD8⁺ TILs are indeed CCR5⁺ CXCR3⁺ and Granzyme B⁺ (not shown), which further indicates that the intra-tumoral expression of the CCR5- and CXCR3-ligands was responsible for recruiting the effector T cells into the tumor.

### Example 3

### Combination of Poly-I:C, IFNα, and COX Inhibitors Selectively Enhances the Production of T_{Eff}-Recruiting Chemokines in Tumor Tissues and Suppresses T_{Reg}-Recruiting Chemokines

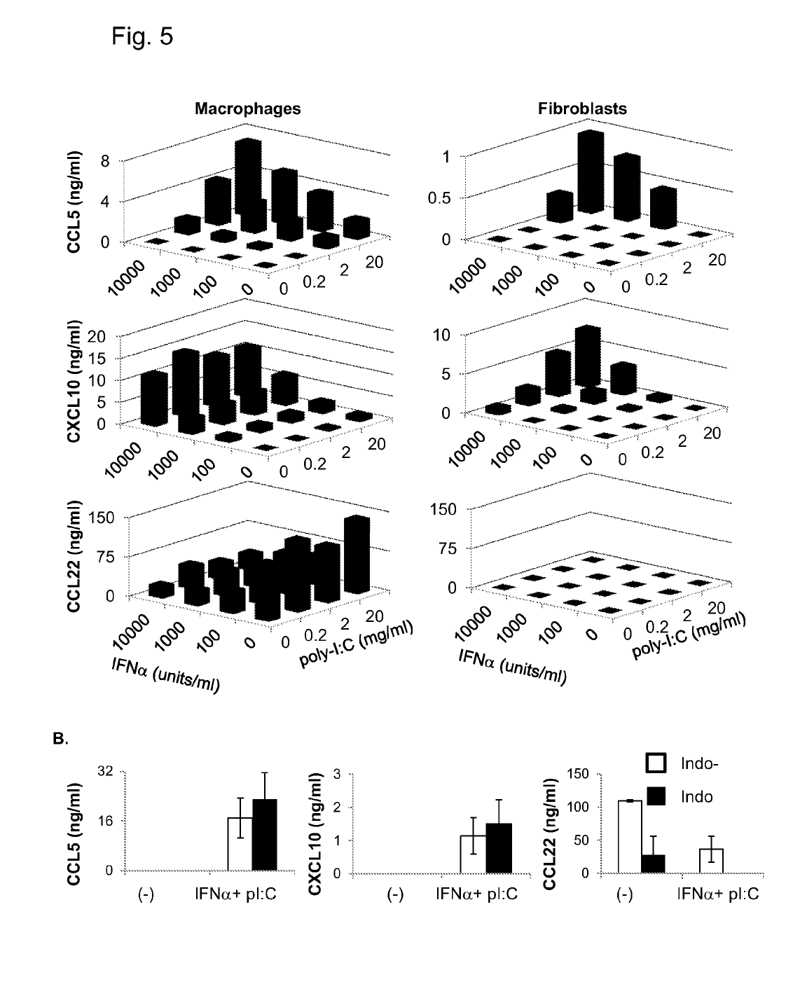

In order to test the possibility of correcting the chemokine environment in the tumors with low ratios of T_{eff}- to T_{reg}-attracting chemokines, we tested in pilot studies the feasibility of modulating their production using different combinations of IFNα, indomethacin (COX1/2 inhibitor) and poly-I:C in individual populations of tumor-relevant cells (known to infiltrate tumors), such as macrophages, or fibroblasts. We observed strong synergy between IFNα and poly-I:C in the induction of CCL5 and CXCL10, and a strong suppressive effect of IFNα on the production of CCL22 in macrophages and fibroblasts (FIG. 5A). These desirable effects were further potentiated in the presence of indomethacin (FIG. 5B).

In order to test the feasibility of using these factors to manipulate the complex microenvironment of whole tumor tissues, involving all the above cell types and their interactions, we used an ex vivo tumor/tissue explant culture system previously developed to study migration of DCs. This system allowed us to avoid nonspecific activation of the chemokine-producing cells during tumor dissociation.

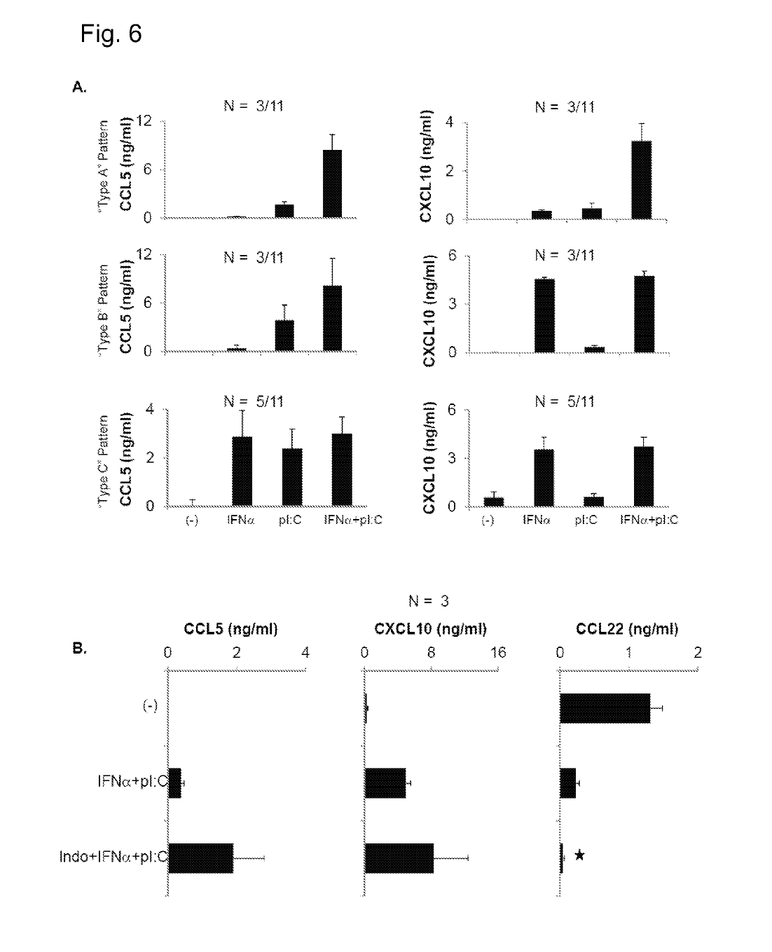

As shown in FIG. 6, different tumor tissues treated with IFNα or poly-I:C alone showed variable chemokine expression, falling into three different patterns: minimal induction of CCL5 and CXCL10; minimal induction of CCL5 but significant induction of CXCL10; or significant induction of both CCL5 and CXCL10 (FIG. 6A). This heterogeneity was observed between tumors from different patients, and even between different lesions within a single patient (FIGS. 6A and 6C). However, combining IFNα and poly-I:C resulted in uniformly high expression of both CCL5 and CXCL10 in all tumors tested (FIGS. 6A and 6C).

Additional exposure to indomethacin (which blocks COX1 and COX2) further enhanced the production of CCL5 and CXCL10 induced by the combination IFNα and poly-I:C and reduced CCL22 levels in whole tumor tissues, with similar results obtained using a selective COX2 blocker (FIGS. 6B and 6D).

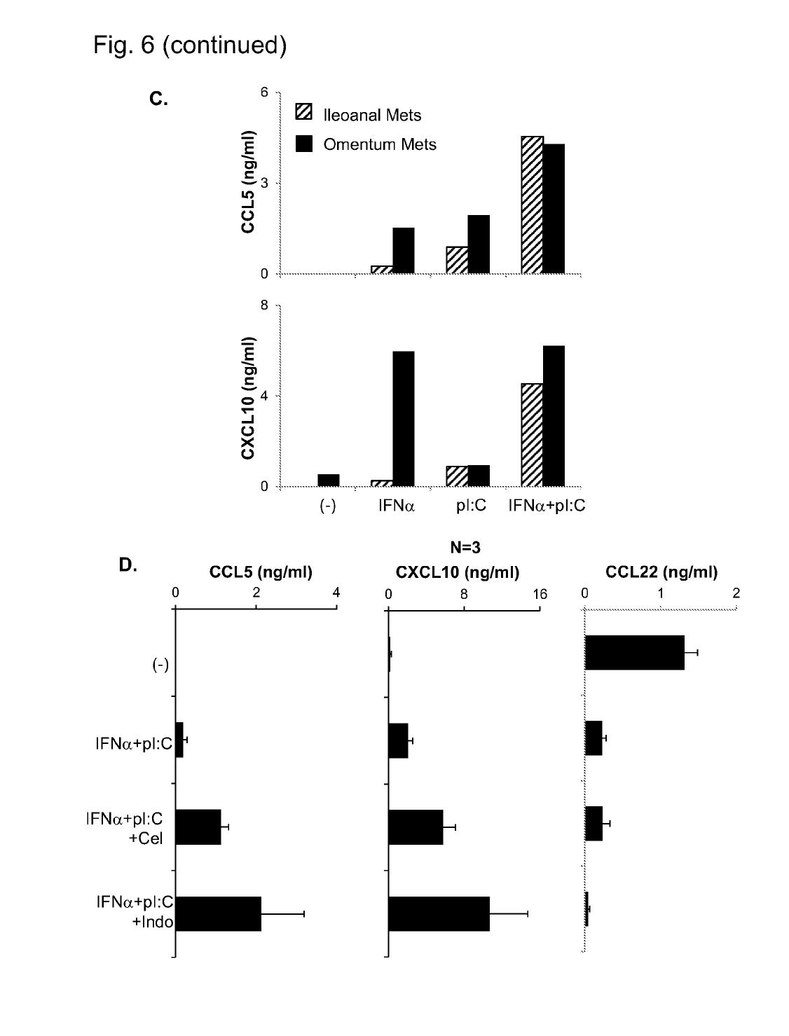

Based on these data, we selected the triple combination of IFNα, poly-I:C and indomethacin as the preferred treatment for all subsequent experiments. This combination consistently enhanced CXCL10 and CCL5 production and suppressed the production of CCL22 in all tumor samples (FIG. 7). Similar observations were also made in case of CXCL9 (data not shown).

The dual staining for HLA-DR (immunohistochemistry) and chemokine mRNA (ISH) demonstrated that CCL22 was expressed predominantly by HLA-DR⁺APCs, while CXCL10 and CCL5 were expressed by both HLA-DR⁺ and HLA-DR⁻ cells (not shown), indicating the contribution of multiple tumor-associated cell types to the production of T_{eff}-recruiting chemokines within the tumor microenvironment.

### Example 4

### Enhanced Activation of Tumor-Associated NF-κB by the Chemokine-Modulatory Regimen Results in Selective or at Least Preferential Induction of CXCL10 in Tumors, Rather than Marginal Healthy Tissues: Feasibility of Tumor-Selective Chemokine Modulation

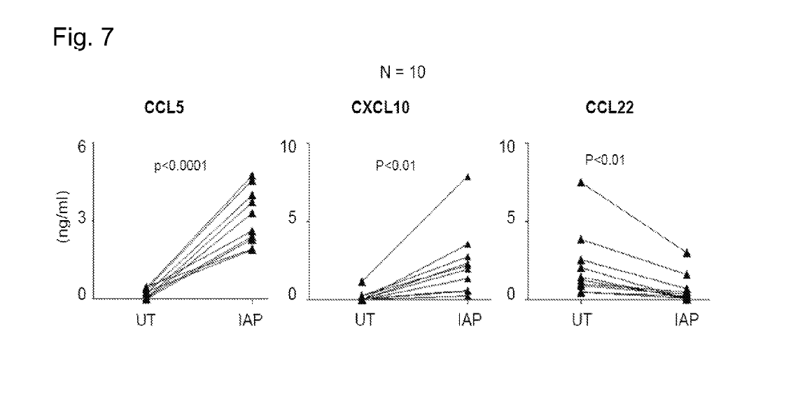

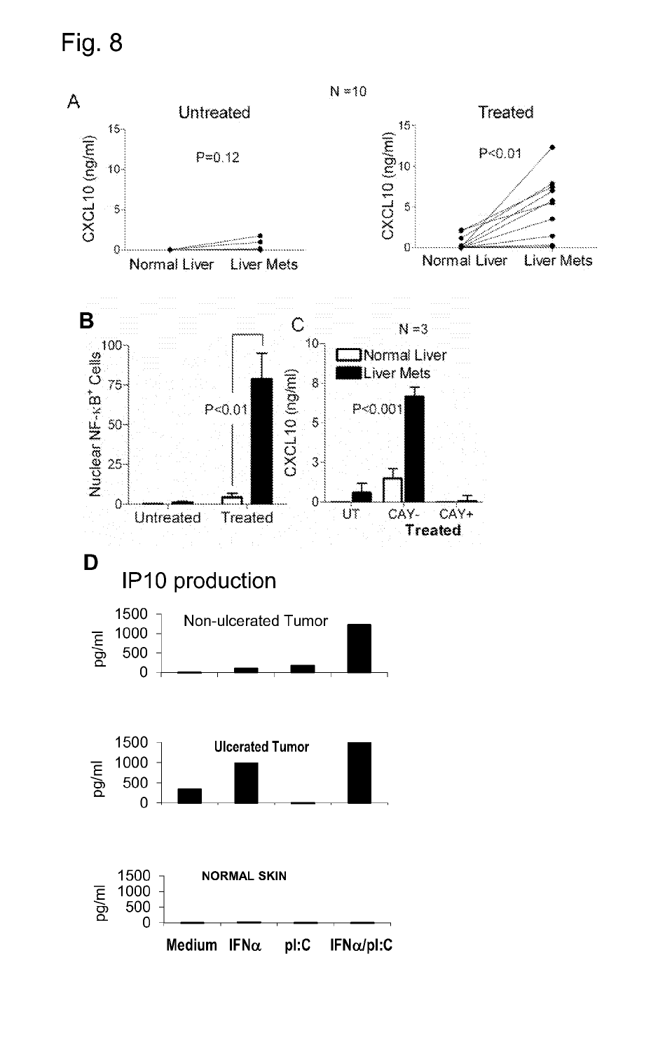

Using matched tissue samples from 10 patients with metastatic colon cancer, we compared the responsiveness to the chemokine-modulating regimen between liver-metastatic tumor tissues and marginal tissues. As shown in FIG. 8 and FIG. 9, while the baseline differences in chemokine production between the untreated liver-metastatic tumors and marginal liver tissues did not reach significance (P=0.12), tumor treatment with the combination of IFNα, poly-I:C and indomethacin induced much more pronounced secretion of CXCL10 by tumor tissues compared to the marginal tissues (P<0.01). Similar observations at the protein and chemokine gene expression level were made in the case of CCL5. This increased responsiveness of tumors compared to marginal tissues was not due to decreased survival of the marginal tissues, as determined by undisturbed expression levels of glycogen phosphorylase (FIG. 9C).

This unexpected selectivity of chemokine modulation was also observed in case of melanoma lesions and adjacent healthy skin, demonstrating the general applicability of our findings (FIG. 8D).

Driven by the previously reported key role of NF-κB in the induction of CXCL10 and other chemokines, and the ubiquitous enhancement of the NF-κB signaling in cancer lesions critically needed for tumor survival and growth, we tested whether potential differences in NF-κB activation could be responsible for the differential ability of the tumors versus marginal tissues to respond to the chemokine modulatory regimen.

In accordance with this possibility, we observed that the colorectal cancer tissues showed not only elevated baseline levels of NF-κB activation (measured by the rate of its nuclear translocation (FIG. 8B), but an even more pronounced ability to further activate NF-κB after the IFNα/poly-I:C/indomethacin treatment (FIG. 8B, right). The key role of NF-κB in CXCL10 production by tumor tissues was validated by using an NF-κB inhibitor, CAY10470, which completely abrogated CXCL10 induction (FIG. 8C).

CCL5 regulation showed a similar pattern (treatment-induced up-regulation in tumors, rather than in marginal tissues) and was also blocked by CAY10470 (Supplementary FIG. S5B), showing the general role of the tumor-associated NF-κB deregulation in the selective induction of T_{eff}-attracting chemokines by the chemokine-modulating regimen. CAY10470, used in these experiments (at 20 μM), was non-toxic, as shown by similar expression of glycogen phosphorylase mRNA in untreated and treated tissues (FIG. 9C).

Interestingly, our confocal microscopy analysis revealed that most of the cells that showed nuclear translocation of NF-κB and produced CCL5 and CXCL10 represented CD45⁺ infiltrating inflammatory cells and tumor-associated fibroblasts, with lesser involvement of the CD326/EpCAM⁺ cancer cells, which produced CCL5 (not shown).

### Example 5

### IFNα/Poly-I:C/Indomethacin-Treated Colorectal Tumors Preferentially Attract Effector CD8⁺ T Cells: Feasibility of Tumor-Selective Attraction of the Spontaneously-Occurring or Vaccination-Induced CTLs to Tumor Lesions, Using the Presented Methods of Chemokine Modulation

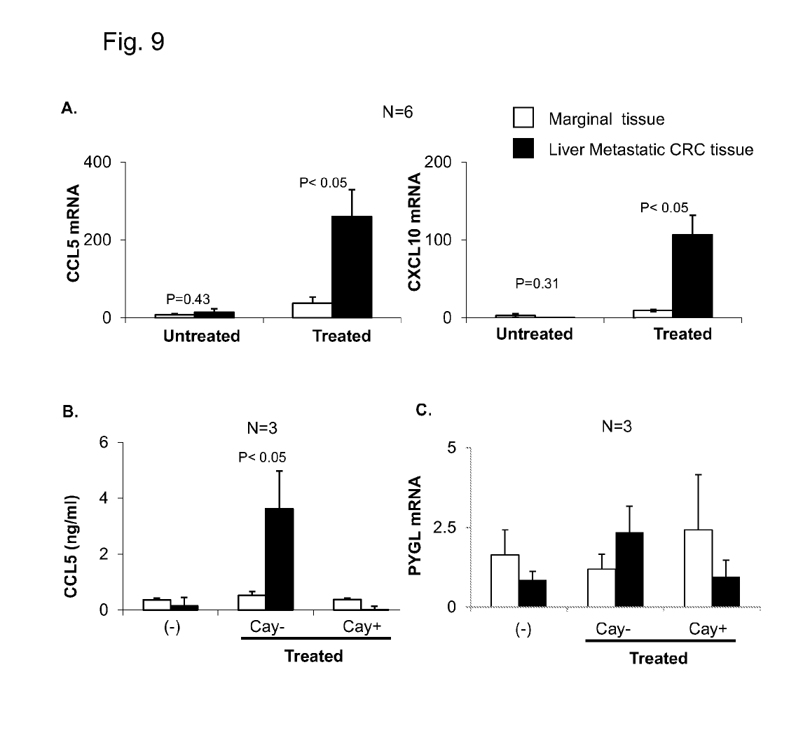

In order to demonstrate that the modulation of chemokine achieved by the combination of IFNα, poly-I:C and indomethacin is indeed sufficient to affect the ability of tumors to attract different subsets of T cells, we used an ex-vivo chemotaxis assay involving the supernatants from differentially-treated tumors and either expanded tumor-infiltrating CD8⁺ T cells (TILs) or polyclonal ex-vivo-induced effector CD8⁺ T cells induced by superantigen-loaded αDC1s. As shown in FIG. 10A-B, both types of effector CD8⁺ T cell showed strongly enhanced migratory responsiveness uniformly to all the IFNα/poly-I:C/indomethacin-treated tumors. In contrast, CD4⁺FOXP3⁺ T cells preferentially migrated to untreated tumors, as determined by Taqman analysis of the migrated blood-isolated CD4⁺ T cells (FIG. 10C), or flow cytometry (not shown).

### Example 6

### Feasibility of Targeting Additional Elements of NF-κB, PGE2/cAMP, and IFN-Signalling Pathways

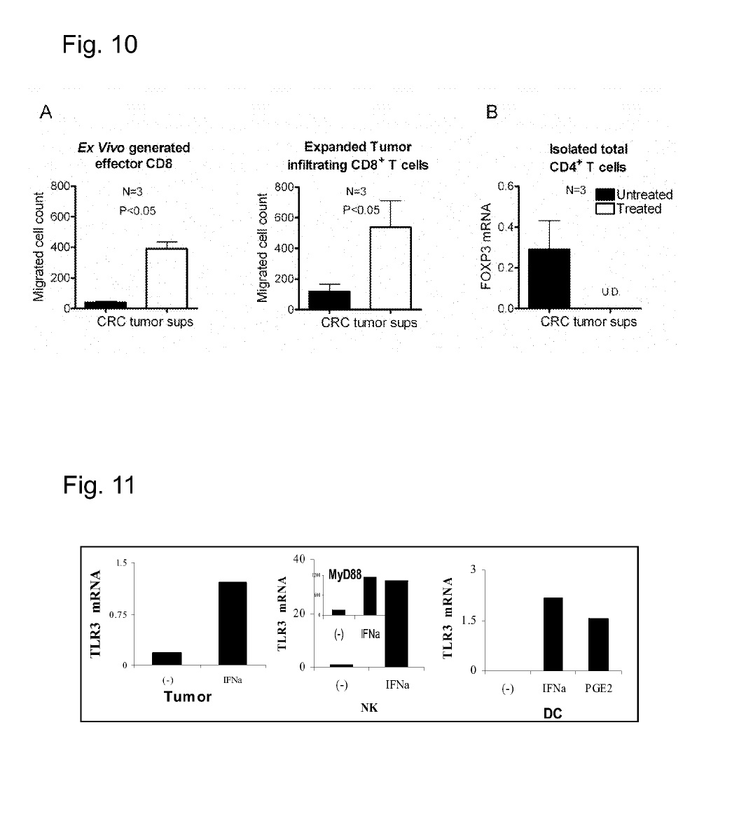

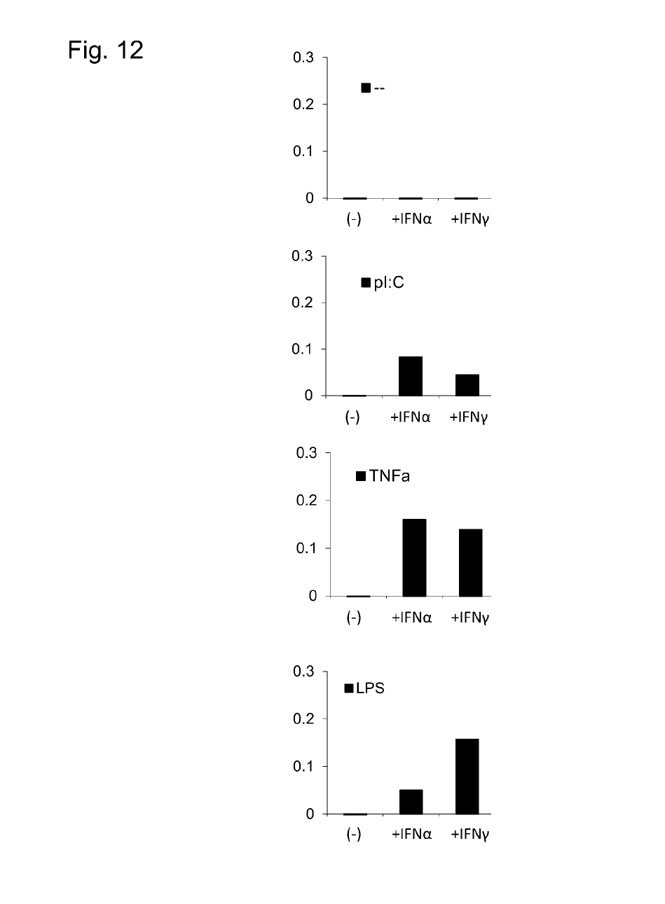

The mechanisms of the synergy between the modulators of the prostaglandin, TLR and interferon systems in the tumor-selective regulation of different classes of chemokines are likely to involve local differences in COX2 activity and PGE receptor expression (EP1-4), different infiltration with TLR3-expressing cells, as well as the regulation of TLR expression and responsiveness as exemplified in FIG. 11. The possibility to use multiple TLR agonists and additional activators of NF-κB (including TNFα) is directly supported by our data presented in FIG. 12.

### Example 7

### Rationale for Targeting Elements of NF-κB, PGE2/cAMP, and IFN-Signalling Pathways During Chemotherapy

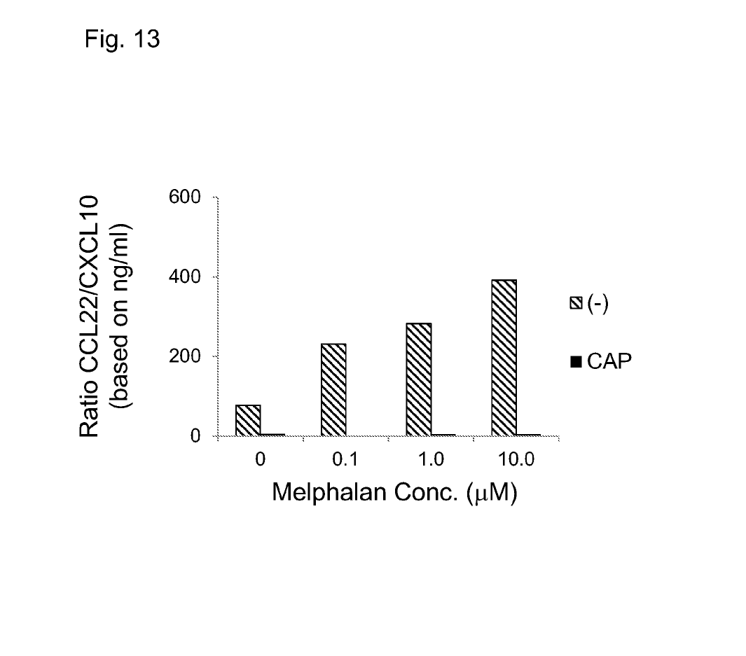

The data presented in FIG. 13 shows an undesirable elevation of the ratio between Treg-attracting and Teff-attracting chemokines (ratio between CCL22/CXCL10) in melanoma tissues treated by a chemotherapeutic agent, melphalan, and the reversal of such undesirable effects of chemotherapy by the combination of celecoxib (COX2 inhibitor), IFNα and poly-I:C. These results provide direct rationale for the incorporation of the presented method in the chemotherapeutic regimen applied to cancer patients and the incorporation of the presented factors in the formulation of chemotherapeutic drugs (combined delivery system; combined formulations of physical linkage).

### VIII. INTERPRETATION OF THE DATA PRESENTED IN EXAMPLES AND FURTHER DISCUSSION

Our data demonstrate the feasibility of tumor-selective modulation of the chemokine environment, using clinically applicable combinations of pharmacologic and biologic factors to correct the balance between tumor-infiltrating T_{eff}- and T_{reg}-cells, the types of immune cells known to differentially affect the clinical course of cancer. Importantly for the clinical application of this strategy, we observed that while the responses of the individual tumor lesions (even in the same patient) to the individual chemokine-modulators were highly variable (consistent with the limited clinical effectiveness of such factors applied individually), the combination of IFNα, poly-I:C and cyclooxygenase inhibitors allowed for highly consistent and selective enhancement of T_{eff}-attracting chemokines (CCL5 and CXCL9-10) within tumor lesions tested, with the concomitant uniform suppression of local CCL22, the T_{reg}-attracting chemokine.

The IFNα/poly-I:C/indomethacin-induced production of T_{eff}-attracting chemokines was highly tumor-selective, suggesting that even systemic administration of these chemokine-modulating factors can preferentially direct effector cells to tumors. While the attraction of different subsets of T cells to different tumor types is known to be regulated by a complex network of additional chemokines not included in our current analysis and can be subject to regulation at the level of chemokine receptor expression, for example by CCR5 polymorphism, our current functional data indicate that the proposed regimen can uniformly promote the influx of effector CD8⁺ T cells (both spontaneously-arising TILs and αDC1 vaccine-induced CTLs). The known role of CXCR3 and CCR5 in the attraction of Th1 cells and NK cells suggests that the proposed regimen may also be able to promote the entry of these additional types of desirable cells into tumors.

We observed that the tumor-selectivity of the proposed regimen depends on the propensity of tumor-associated fibroblasts and infiltrating inflammatory cells (with lesser involvement of tumor cells themselves) to not only spontaneously hyper-activate NF-κB, but also respond to treatment with further-enhanced levels of NF-κB activation. Since NF-κB activation, critically involved in tumor survival and growth, represents an intrinsic feature of many tumor types, the current data suggest that the currently-described NF-κB-targeting modulation of the tumor microenvironment may be applicable to multiple types of cancer.

Our analyses performed so far did not reveal any differences between the expression of the IFNα receptor, TLR3, IRF1, or IRF3 between tumors and marginal tissues (data not shown), but our current work focuses on the differential regulation of each of the pathways (poly-I:C, IFNα and PGE₂ responsiveness) in whole tumor tissues and different types of tumor-associated cells. Similarly, we are also evaluating the mechanisms underlying the increased sensitivity of tumor-related cells to activate NF-κB and the relative heterogeneity of different tumors with regard to the requirement for poly-I:C activation, which may help us to identify new strategies of chemokine regulation and of targeting NF-κB in tumor therapy.

### DESCRIPTION OF PREFERRED EMBODIMENTS

The following is a description of some preferred embodiments of a method of the modulation of tumor microenvironment (or other tissue of therapeutic interest), targeting TLRs (or the receptors of other known NF-κB activators), prostaglandin/cAMP system and interferons and interferon signaling:

One preferred embodiment is the administration to a patient with cancer, precancerous lesion or previously treated cancer, a therapeutically effective amount of at least two different agents that act synergistically to selectively induce the production of effector cell-attracting chemokines, such as IP-10 (CXCL10), and RANTES (CCL5) in tumor tissues (or other disease-effected tissues), while suppress or have no impact on the production of CCL22, the chemokine known to attract undesirable regulatory T cells.

In case of prevention or treatment of cancer, some premalignant states and many infections, a preferred embodiment of this invention is a combined application of a TLR ligand or another activator of the NF-κB pathway with an inhibitor of prostanoids (or with an inhibitor of prostaglandin receptors and/or inhibitors of alternative cAMP-elevating agents or inhibitors of cAMP signaling) and with prior, concomitant or subsequent administration of IFNα and/or other type I or type II interferons), in order to selectively enhance the production of the T_{eff}-attracting chemokines while suppressing the production of T_{reg}-attracting chemokines.

In case of prevention or treatment of the diseases states associated with undesirable over-activation of the immune system (chronic inflammations, some premalignant states, autoimmune phenomena, or transplant rejection, including the rejection of transplanted organs, tissues, and isolated cells, including transplant rejection and graft-versus-host (GvH) disease), a preferred embodiment of this invention is a combined application of a TLR ligand or other activators of NF-κB pathways with prostanoids or other cAMP-elevating agents (and with potential additional use of inhibitors of IFN production of IFN responsiveness), in order to selectively enhance the production of the T_{reg}-attracting chemokines while suppressing the production of T_{eff}-attracting chemokines.

In some embodiments, methods are provided for treating cancer or preventing cancer's occurrence or recurrence in a subject include administering to the subject at therapeutically effective amount of a prostaglandin inhibitor or other cAMP suppressing agent that increases IP-10/CXCL10 production and inhibits MDC/CCL22 production and a therapeutically effective amount of a Toll-like receptor (TLR) agonist.

In other embodiments, methods are provided for treating cancer or preventing cancer's occurrence or recurrence in a subject by administering to the subject a therapeutically effective amount of an interferon or an agent that increases IP-10 activity and a therapeutically effective amount of a prostaglandin synthesis inhibitor, thereby treating or preventing colorectal cancer in the subject.

An embodiment of this invention is administering to the subject at therapeutically effective amounts of (1) a Toll-like receptor (TLR) agonist, combined with (2) a blocker of prostaglandin synthesis, a blocker of PGE2 receptor or a blocker of cAMP signaling and/or (3) therapeutically effective amount of an interferon, applied simultaneously of sequentially, using a common delivery system or a combination of delivery systems allowing the release of each of the factors with different kinetics.

Another embodiment of this invention is administering to the subject at a therapeutically effective amounts of a complex molecule incorporating (1) a Toll-like receptor (TLR) agonist, (2) a blocker of prostaglandin synthesis, a blocker of PGE2 receptor or a blocker of cAMP signaling and/or (3) an interferon or an agonist of the type I or type II interferon receptor (an antibody or a small molecule. A related embodiment is the application of the two or three of the above factors using a common medium (emulsion, liposomes, nanovesicles, slow release matrix, a porous material.

Another embodiment of this invention is administering to the subject at a therapeutically effective amounts of a complex molecule incorporating (1) a Toll-like receptor (TLR) agonist, (2) a blocker of prostaglandin synthesis, a blocker of PGE2 receptor or a blocker of cAMP signaling and/or (3) an interferon or an agonist of the type I or type II interferon receptor (an antibody or a small molecule), sequentially or simultaneously through the same catheter.

Another embodiment of this invention is administering to the subject at a therapeutically effective amounts of a complex molecule incorporating (1) a Toll-like receptor (TLR) agonist, (2) a blocker of prostaglandin synthesis, a blocker of PGE2 receptor or a blocker of cAMP signaling and/or (3) an interferon or an agonist of the type I or type II interferon receptor (an antibody or a small molecule), sequentially or simultaneously using an implantable pump or two or more pumps.

Since not only the spontaneously arising tumor-specific effector cells (or effector cells against infectious agents), but also tumor-specific effector cells induced by different cancer vaccines, including alpha-DC1s or other type of type-1-polarized DCs (such as induced by the combination of LPS and IFNγ or by the combination of TNFα and IFNγ), express high levels of CCR5 or CXCR3 on tumor-specific T cells, the tumor-selective induction of CCR5 ligands or/and CXCR3 ligands in tumor tissues may be particularly effective in combination with the application of such vaccines.

In case of prevention or treatment of the diseases associated with the undesirable over-activation of the immune system (chronic inflammations, some premalignant states, autoimmune phenomena, or transplant rejection, including the rejection of transplanted organs, tissues, and isolated cells, including transplant rejection and graft-versus-host (GvH) disease), a preferred embodiment of this invention is combined application (concomitant or sequential) of TLR ligands or other activators of NF-κB pathways with prostanoids or other cAMP-elevating agents (and with potential inhibitors of IFN production of IFN responsiveness), using a common medium (emulsion, liposomes, nanovesicles, slow release matrix, a porous material), same catheter, an implantable pump or several pumps, or in a physically linked form.

Furthermore, the embodiments of the current invention also include:
Tumor-selective (preferential for the tumor tissue, rather than healthy tissues), chemokine modulation as a stand-alone treatment to boost the naturally-occurring immunity against cancer or infections.
Tumor-selective chemokine modulation as a way to boost the ability of vaccination-induced T cells (or adoptively transferred T cells) against cancer or infectious agents to enter tumor tissues and to mediate cancer regression or stabilization.
Pathogen-specific (matched to the pathogen-affected tissue) and tumor-selective (preferential for the affected tissue, rather than healthy tissues), chemokine modulation as a stand-alone treatment to boost the naturally-occurring immunity against infections.
Reciprocal approaches aimed at boosting the Treg infiltration and suppression of Teff-attracting CKs may be applied to transplantation, autoimmune diseases, or some chronic inflammatory processes.

It will be apparent that the precise details of the methods or compositions described may be varied or modified without departing from the spirit of the described invention. We claim all such modifications and variations that fall within the scope and spirit of the claims below.

1. A method for treating cancer or preventing cancer occurrence or recurrence in a subject, comprising administering to the subject therapeutically effective amounts of (1) an NF-κB activator, (2) a blocker of prostaglandin synthesis or a blocker of prostaglandin responsiveness, and (3) an interferon.

2. A method for treating cancer or preventing cancer occurrence or recurrence in a subject, comprising administering to the subject therapeutically effective amounts of (1) a Toll-like receptor (TLR) agonist, (2) a blocker of prostaglandin synthesis or a blocker of prostaglandin responsiveness, and (3) an interferon.

3. A method for treating cancer or preventing cancer occurrence or recurrence in a subject, comprising administering to the subject therapeutically effective amounts of (1) an NF-κB activator and (2) a blocker of prostaglandin synthesis or a blocker of prostaglandin responsiveness.

4. A method for treating cancer or preventing cancer occurrence or recurrence in a subject, comprising administering to the subject therapeutically effective amounts of (1) a Toll-like receptor (TLR) agonist and (2) a blocker of prostaglandin synthesis or a blocker of prostaglandin responsiveness.

5. A method for treating cancer or preventing cancer occurrence or recurrence in a subject, comprising administering to the subject therapeutically effective amounts of (1) an NF-κB activator and (2) an interferon.

6. A method for treating cancer or preventing cancer occurrence or recurrence in a subject, comprising administering to the subject therapeutically effective amounts of (1) a Toll-like receptor (TLR) agonist and (2) an interferon.

7. A method for treating or preventing the onset or recurrence of an autoimmune disease, chronic inflammatory disease, transplant rejection, or GvH, comprising of administering to the subject therapeutically effective amounts of (1) an NF-κB activator and (2) a prostaglandin or other activator of the cAMP-signaling pathway.

8. A method for treating or preventing the onset or recurrence of an autoimmune disease, chronic inflammatory disease, transplant rejection, or GvH, comprising of administering to the subject therapeutically effective amounts of (1) a TLR ligand and (2) a prostaglandin or other activator of the cAMP-signaling pathway.

9. A method for treating cancer or preventing cancer occurrence or recurrence in a subject, comprising administering to the subject therapeutically effective amounts of (1) an NF-κB activator, (2) a blocker of prostaglandin synthesis or a blocker of prostaglandin responsiveness, (3) an interferon and (4) a chemotherapeutic agent.

10. A method for treating cancer or preventing cancer occurrence or recurrence in a subject, comprising administering to the subject therapeutically effective amounts of (1) a Toll-like receptor (TLR) agonist (2) a blocker of prostaglandin synthesis or a blocker of prostaglandin responsiveness, (3) an interferon and (4) a chemotherapeutic agent.

11. A method for treating cancer or preventing cancer occurrence or recurrence in a subject, comprising administering to the subject therapeutically effective amounts of (1) an NF-κB activator (2) a blocker of prostaglandin synthesis or a blocker of prostaglandin responsiveness, and (3) a chemotherapeutic agent.

12. A method for treating cancer or preventing cancer occurrence or recurrence in a subject, comprising administering to the subject therapeutically effective amounts of (1) a Toll-like receptor (TLR) agonist (2) a blocker of prostaglandin synthesis or a blocker of prostaglandin responsiveness, and (3) a chemotherapeutic agent.

13. A method for treating cancer or preventing cancer occurrence or recurrence in a subject, comprising administering to the subject therapeutically effective amounts of (1) an NF-κB activator, (2) an interferon and (3) a chemotherapeutic agent.

14. A method for treating cancer or preventing cancer occurrence or recurrence in a subject, comprising administering to the subject therapeutically effective amounts of (1) a Toll-like receptor (TLR) agonist, (2) an interferon, and (3) a chemotherapeutic agent.

15. A method for treating cancer or preventing cancer occurrence or recurrence in a subject, comprising administering to the subject therapeutically effective amounts of (1) an NF-κB activator, (2) a blocker of prostaglandin synthesis or a blocker of prostaglandin responsiveness, (3) an interferon and (4) a vaccine or other therapy for enhancing the expression levels of CCR5 or CXCR3 on tumor-specific T cells.

16. A method for treating cancer or preventing cancer occurrence or recurrence in a subject, comprising administering to the subject therapeutically effective amounts of (1) a Toll-like receptor (TLR) agonist (2) a blocker of prostaglandin synthesis or a blocker of prostaglandin responsiveness, (3) an interferon and (4) a vaccine or other therapy for enhancing the expression levels of CCR5 or CXCR3 on tumor-specific T cells.

17. A method for treating cancer or preventing cancer occurrence or recurrence in a subject, comprising administering to the subject therapeutically effective amounts of (1) an NF-κB activator (2) a blocker of prostaglandin synthesis or a blocker of prostaglandin responsiveness, and (3) a vaccine or other therapy for enhancing the expression levels of CCR5 or CXCR3 on tumor-specific T cells.

18. A method for treating cancer or preventing cancer occurrence or recurrence in a subject, comprising administering to the subject therapeutically effective amounts of (1) a Toll-like receptor (TLR) agonist (2) a blocker of prostaglandin synthesis or a blocker of prostaglandin responsiveness, and (3) a vaccine or other therapy for enhancing the expression levels of CCR5 or CXCR3 on tumor-specific T cells.

19. A method for treating cancer or preventing cancer occurrence or recurrence in a subject, comprising administering to the subject therapeutically effective amounts of (1) an NF-κB activator, (2) an interferon and (3) a vaccine or other therapy for enhancing the expression levels of CCR5 or CXCR3 on tumor-specific T cells.

20. A method for treating cancer or preventing cancer occurrence or recurrence in a subject, comprising administering to the subject therapeutically effective amounts of (1) a Toll-like receptor (TLR) agonist, (2) an interferon, and (3) a vaccine or other therapy for enhancing the expression levels of CCR5 or CXCR3 on tumor-specific T cells.

21. The method of claim 2, 4, 6, 8, 10, 12, 14, 16, 18 or 20, wherein the TLR agonist is a TLR-3 ligand.

22. The method of claim 21, wherein the TLR-3 ligand is poly I:C.

23. The method of claim 21, wherein the TLR-3 ligand is a derivative of poly I:C, capable of binding TLR3.

24. The method of claim 21, wherein the TLR-3 ligand is a double-stranded RNA, capable of binding TLR3.

25. The method of claim 21, wherein the TLR-3 ligand is a small molecule.

26. The method of claim 21, wherein the TLR-3 ligand is an antibody.

27. The method of claim 2, 4, 6, 8, 10, 12, 14, 16, 18 or 20, wherein the TLR agonist is a TLR-4 ligand.

28. The method of claim 2, 4, 6, 8, 10, 12, 14, 16, 18 or 20, wherein the TLR agonist is a TLR-7 ligand.

29. The method of claim 2, 4, 6, 8, 10, 12, 14, 16, 18 or 20, wherein the TLR agonist is a TLR-8 ligand.

30. The method of claim 2, 4, 6, 8, 10, 12, 14, 16, 18 or 20, wherein the TLR agonist is a TLR-9 ligand.

31. The method of claim 2, 4, 6, 8, 10, 12, 14, 16, 18 or 20, wherein the TLR agonist is a TLR-5 ligand.

32. The method of claim 2, 4, 6, 8, 10, 12, 14, 16, 18 or 20, wherein the TLR agonist is a TLR-2 ligand.

33. The method of claim 2, 4, 6, 8, 10, 12, 14, 16, 18 or 20, wherein the TLR agonist is a TLR-1 ligand.

34. The method of claim 1, 3, 5, 7, 9, 11, 13, 15, 17 or 19, wherein the NF-κB activator is TNFα, IL-1β or other proinflammatory cytokine or NF-κB-activating endogenous or exogenous ligand of a pattern recognition receptor.

35. The method of claim 1, 2, 3, 4, 9, 10, 11, 12, 15, 16, 17 or 18, wherein the blocker of prostaglandin synthesis is a cyclooxygenase (COX) inhibitor.

36. The method of claim 1, 2, 3, 4, 9, 10, 11, 12, 15, 16, 17 or 18, wherein the blocker of prostaglandin synthesis is an inhibitor of both cyclooxygenase (COX)-1 and cyclooxygenase (COX)-2.

37. The method of claim 1, 2, 3, 4, 9, 10, 11, 12, 15, 16, 17 or 18, wherein the blocker of prostaglandin synthesis is a selective inhibitor of cyclooxygenase 2 (COX-2) inhibitor.

38. The method of claim 1, 2, 3, 4, 9, 10, 11, 12, 15, 16, 17 or 18, wherein the blocker of prostaglandin synthesis is celecoxib.

39. The method of claim 1, 2, 3, 4, 9, 10, 11, 12, 15, 16, 17 or 18, wherein the blocker of prostaglandin synthesis is rofecoxib.

40. The method of claim 1, 2, 3, 4, 9, 10, 11, 12, 15, 16, 17 or 18, wherein the blocker of prostaglandin synthesis is indomethacin.

41. The method of claim 1, 2, 3, 4, 9, 10, 11, 12, 15, 16, 17 or 18, wherein the blocker of prostaglandin responsiveness is an inhibitor of prostaglandin E2 receptors EP2 or EP4.

42. The method of claim 1, 2, 5, 6, 9, 10, 13, 14, 15, 16, 19 or 20, wherein the interferon is a type-1 interferon.

43. The method of claim 1, 2, 5, 6, 9, 10, 13, 14, 15, 16, 19 or 20, wherein the interferon is an interferon alpha or interferon beta.

44. The method of claim 1, 2, 5, 6, 9, 10, 13, 14, 15, 16, 19 or 20, wherein the interferon is interferon gamma.

45. The method of claim 15, 16, 17, 18, 19 or 20, wherein the vaccine comprises alpha-DC1s.

46. The method of claim 15, 16, 17, 18, 19 or 20, wherein the vaccine comprises any type of type-1-polarized DCs such as those that are induced by the combination of LPS and IFNγ or by the combination of TNFα and IFNγ.

47. The method of claim 7 or 8, wherein the prostaglandin is prostaglandin E2 (PGE2).

48. The method of claim 7 or 8, wherein the cAMP-elevating agent is an agonist of the EP2 or EP4 receptors of PGE2.

49. The method of claim 7 or 8, wherein the cAMP-elevating agent is histamine, adrenaline, noradrenaline, or an agonist of a cAMP-elevating receptor of histamine, adrenaline, or noradrenaline.

50. The method of claim 7 or 8, wherein the activator of the cAMP-signaling pathway is an activator of the adelylate cyclase, CREB or a downstream element of CREB signaling pathway.

51. The method of claim 1, 2, 3 or 4, wherein the blocker of prostaglandin responsiveness is an inhibitor of cAMP synthesis, an inducer or phosphodiesterase activity or other promoter of cAMP degradation, or an inhibitor of CREB or a downstream element of CREB signaling pathway.
